(19) United States
(12) Patent Application Publication
(10) Pub. No.: US 2007/0191323 A1
(43) Pub. Date: Aug. 16, 2007

(54) STABLE CORTICOSTEROID MIXTURES

(75) Inventors: Malcolm Hill, Solana Beach, CA (US); Cynthia LiCalsi, San Diego, CA (US)

Correspondence Address: WILSON SONSINI GOODRICH & ROSATI, 650 PAGE MILL ROAD, PALO ALTO, CA, 94304-1050, US

(73) Assignee: VERUS PHARMACEUTICALS, INC., San Diego, CA (US)

(21) Appl. No.: 11/675,575

(22) Filed: Feb. 15, 2007

Related U.S. Application Data
(60) Provisional application No. 60/774,152, filed on Feb. 15, 2006.
(60) Provisional application No. 60/774,073, filed on Feb. 15, 2006.
(60) Provisional application No. 60/774,151, filed on Feb. 15, 2006.

(51) Int. Cl. A61K 31/573; A61K 31/56

(52) U.S. Cl. 514/171; 540/63; 552/503

(57) ABSTRACT

A corticosteroid mixture, such as a budesonide solution, is prepared by the active and inactive ingredients of the mixture under oxygen-depleted conditions. The resulting mixture demonstrates superior stability of the active pharmaceutical ingredient corticosteroid. The invention provides novel methods of manufacturing corticosteroid mixtures, wherein the resulting mixtures possess superior stability as compared to known methods.

### PRIORITY CLAIM AND CROSS-REFERENCE TO RELATED APPLICATIONS

This application claims the benefit of and priority under 35 U.S.C. 119(e) to U.S. provisional patent application 60/774,073, filed on Feb. 15, 2006, which is incorporated herein by reference in its entirety. This application further claims the benefit of and priority under 35 U.S.C. §119(e) from U.S. Provisional Patent Application No. 60/774,151, which was filed on Feb. 15, 2006, and which is incorporated herein by reference in its entirety. This application further claims the benefit of and priority under 35 U.S.C. §119(e) from U.S. Provisional Patent Application No. 60/774,152, filed on Feb. 15, 2006, which is incorporated herein by reference in its entirety.

This application is related to copending application Ser. No. 11/675,563, filed Feb. 15, 2007, entitled “Sterilization of Corticosteroids With Reduced Mass Loss,” Attorney Docket Number 31622-717/201, which is incorporated herein by reference in its entirety. This application is related to copending application Ser. No. 11/675,569, filed Feb. 15, 2007, entitled “Methods of Manufacturing Corticosteroid Solutions,” Attorney Docket Number 31622-718/201, which is incorporated herein by reference in its entirety.

### BACKGROUND OF THE INVENTION

Aqueous solutions of budesonide have been reported. See, for example, WO 2005/065649, WO 2005/065435 and WO 2005/065651 mention budesonide solutions comprising, as a solubility enhancer, SEB7-β-CD (Captisol®)(CyDex). Although these applications teach purging a filtered budesonide solution with nitrogen gas under certain circumstances, the stability of the resulting budesonide solution is such that it would be desirable to further enhance the stability of the solution.

There is thus a need in the art for a method of preparing a method of making a stabilized corticosteroid composition. There is further a need for a stabilized corticosteroid composition.

### SUMMARY OF THE INVENTION

The foregoing and further needs are met by embodiments of the invention, which provide a novel process of preparing a corticosteroid mixture. The process includes mixing ingredients of the corticosteroid mixture in a mixing vessel under oxygen-depleted conditions. The thus-produced corticosteroid mixture has enhanced stability. In some preferred embodiments, the mixture is a corticosteroid solution, which optionally comprises one or more additional ingredients. Optional additional ingredients include solubility enhancers, especially cyclodextrin solubility enhancers, such as a sulfoalkyl ether cyclodextrin (SAE-CD), especially SBE7-β-CD. In some embodiments, corticosteroid solutions of the invention demonstrate less than 10% loss of corticosteroid potency after 24 months under normal conditions (25° C. and 60% relative humidity). In some embodiments, budesonide solutions of the invention demonstrate less than 10% loss of budesonide potency after 24 months under normal conditions (25° C. and 60% relative humidity). As used herein, the term “potency” refers to the concentration of the corticosteroid (e.g. budesonide) in solution.

The foregoing and other needs are further met by embodiments of the invention, which provide a corticosteroid mixture which, after exposing the corticosteroid solution to accelerated conditions of 40° C. and 75% relative humidity for 3 months, demonstrates no more than about 2% degradation of the corticosteroid in the mixture. In some preferred embodiments, the mixture is a corticosteroid solution, which optionally comprises one or more additional ingredients. Optional additional ingredients include solubility enhancers, especially cyclodextrin solubility enhancers, such as a sulfoalkyl ether cyclodextrin (SAE-CD), especially SBE7-β-CD. In some embodiments, corticosteroid solutions of the invention demonstrate no more than 10% loss of corticosteroid potency after 12 months at accelerated conditions (40° C. and 75% relative humidity). In some embodiments, budesonide solutions of the invention demonstrate no more than 10% loss of budesonide potency after 12 months at accelerated conditions (40° C. and 75% relative humidity).

The foregoing and other needs are further met by embodiments of the invention, which provide a process of preparing a corticosteroid mixture, comprising mixing ingredients of the corticosteroid mixture in a mixing vessel under oxygen-depleted conditions to produce the corticosteroid mixture, wherein the ingredients include as starting materials corticosteroid and water, wherein the corticosteroid mixture, upon exposing the corticosteroid mixture to normal or accelerated conditions (e.g., 30° C., 40° C. or 60° C.) for a period of 12 months or more demonstrates about 0.1% to about 5%, about 0.2% to about 4%, about 0.5% to about 3%, about 0.7% to about 2%, about 0.8% to about 2% or about 1 to about 2%, less than about 10%, less than about 7.5% less than about 5%, less than about 4%, less than about 3%, less than about 2.5%, less than about 2.2% or about 2% or less degradation. In some preferred embodiments, the mixture is a corticosteroid solution, which optionally comprises one or more additional ingredients. Optional additional ingredients include solubility enhancers, especially cyclodextrin solubility enhancers, such as a sulfoalkyl ether cyclodextrin (SAE-CD), especially SBE7-β-CD. In some embodiments, corticosteroid solutions of the invention demonstrate less than 10% loss of corticosteroid potency after 24 months under normal conditions (25° C. and 60% relative humidity). In some embodiments, budesonide solutions of the invention demonstrate less than 10% loss of budesonide potency after 24 months under normal conditions (25° C. and 60% relative humidity).

The foregoing and other needs are further met by embodiments of the invention, which provide a process of preparing a budesonide mixture, comprising mixing ingredients of the budesonide mixture in a mixing vessel under oxygen-depleted conditions to produce the budesonide mixture, wherein the ingredients include as starting materials budesonide and water, wherein the budesonide mixture, upon exposing the budesonide mixture to normal or accelerated conditions (e.g., 30° C., 40° C. or 60° C.) for a period of 12 months or more demonstrates about 0.1% to about 5%, about 0.2% to about 4%, about 0.5% to about 3%, about 0.7% to about 2%, about 0.8% to about 2% or about 1 to about 2%, less than about 10%, less than about 7.5% less than about 5%, less than about 4%, less than about 3%, less than about 2.5%, less than about 2.2% or about 2% or less degradation. In some preferred embodiments, the mixture is a budesonide solution, which optionally comprises one or more additional ingredients. Optional additional ingredients include solubility enhancers, especially cyclodextrin solubility enhancers, such as a sulfoalkyl ether cyclodextrin (SAE-CD), especially SBE7-β-CD. In some embodiments, corticosteroid solutions of the invention demonstrate less than 10% loss of corticosteroid potency after 24 months under normal conditions (25° C. and 60% relative humidity). In some embodiments, budesonide solutions of the invention demonstrate less than 10% loss of budesonide potency after 24 months under normal conditions (25° C. and 60% relative humidity).

The foregoing and other needs are further met by embodiments of the invention, which provide a process of preparing a corticosteroid mixture, comprising mixing ingredients of the corticosteroid mixture in a mixing vessel under oxygen-depleted conditions to produce the corticosteroid mixture, wherein the ingredients include as starting materials corticosteroid and water, wherein the corticosteroid mixture, upon exposing the corticosteroid mixture to normal patient storage conditions for a period of 6 months or more demonstrates about 0.1% to about 5%, about 0.2% to about 4%, about 0.5% to about 3%, about 0.7% to about 2%, about 0.8% to about 2% or about 1 to about 2%, less than about 10%, less than about 7.5% less than about 5%, less than about 4%, less than about 3%, less than about 2.5%, less than about 2.2% or about 2% or less degradation. In some preferred embodiments, the mixture is a corticosteroid solution, which optionally comprises one or more additional ingredients. Optional additional ingredients include solubility enhancers, especially cyclodextrin solubility enhancers, such as a sulfoalkyl ether cyclodextrin (SAE-CD), especially SBE7-β-CD. In some embodiments, corticosteroid solutions of the invention demonstrate less than 10% loss of corticosteroid potency after 24 months under normal conditions (25° C. and 60% relative humidity). In some embodiments, budesonide solutions of the invention demonstrate less than 10% loss of budesonide potency after 24 months under normal conditions (25° C. and 60% relative humidity).

The foregoing and other needs are further met by embodiments of the invention, which provide a process of preparing a corticosteroid mixture, comprising mixing ingredients of the corticosteroid mixture in a mixing vessel under oxygen-depleted conditions to produce the corticosteroid mixture, wherein the ingredients include as starting materials corticosteroid and water, wherein the corticosteroid mixture, upon exposing the corticosteroid mixture to normal or accelerated conditions (e.g., 30° C., 40° C. or 60° C.) for a period of 12 months or more demonstrates about 0.1% to about 5%, about 0.2% to about 4%, about 0.5% to about 3%, about 0.7% to about 2%, about 0.8% to about 2% or about 1 to about 2%, less than about 10%, less than about 7.5% less than about 5%, less than about 4%, less than about 3%, less than about 2.5%, less than about 2.2% or about 2% or less degradation. In some preferred embodiments, the mixture is a corticosteroid solution, which optionally comprises one or more additional ingredients. Optional additional ingredients include solubility enhancers, especially cyclodextrin solubility enhancers, such as a sulfoalkyl ether cyclodextrin (SAE-CD), especially SBE7-β-CD. In some embodiments, corticosteroid solutions of the invention demonstrate less than 10% loss of corticosteroid potency after 24 months under normal conditions (25° C. and 60% relative humidity). In some embodiments, budesonide solutions of the invention demonstrate less than 10% loss of budesonide potency after 24 months under normal conditions (25° C. and 60% relative humidity).

The foregoing and other needs are further met by embodiments of the invention, which provide a process of preparing a corticosteroid mixture, comprising mixing ingredients of the corticosteroid mixture in a mixing vessel under oxygen-depleted conditions to produce the corticosteroid mixture, wherein the ingredients include as starting materials corticosteroid and water, wherein the corticosteroid mixture, upon exposing the corticosteroid mixture to normal or accelerated conditions (e.g. 30° C., 40° C. or 60° C.) for a period of 24 months or more demonstrates about 0.1% to about 5%, about 0.2% to about 4%, about 0.5% to about 3%, about 0.7% to about 2%, about 0.8% to about 2% or about 1 to about 2%, less than about 10%, less than about 7.5% less than about 5%, less than about 4%, less than about 3%, less than about 2.5%, less than about 2.2% or about 2% or less degradation. In some preferred embodiments, the mixture is a corticosteroid solution, which optionally comprises one or more additional ingredients. Optional additional ingredients include solubility enhancers, especially cyclodextrin solubility enhancers, such as a sulfoalkyl ether cyclodextrin (SAE-CD), especially SBE7-β-CD. In some embodiments, corticosteroid solutions of the invention demonstrate less than 10% loss of corticosteroid potency after 24 months under normal conditions (25° C. and 60% relative humidity). In some embodiments, budesonide solutions of the invention demonstrate less than 10% loss of budesonide potency after 24 months under normal conditions (25° C. and 60% relative humidity).

The foregoing and other needs are further met by embodiments of the invention, which provide a corticosteroid mixture which, upon exposing the corticosteroid mixture to normal or accelerated conditions (e.g. 30° C., 40° C. or 60° C.) for period of 6 weeks or more demonstrates about 0.1% to about 5%, about 0.2% to about 4%, about 0.5% to about 3%, about 0.7% to about 2%, about 0.8% to about 2% or about 1 to about 2%, less than about 10%, less than about 7.5% less than about 5%, less than about 4%, less than about 3%, less than about 2.5%, less than about 2.2% or about 2% or less degradation. In some preferred embodiments, the mixture is a corticosteroid solution, which optionally comprises one or more additional ingredients. Optional additional ingredients include solubility enhancers, especially cyclodextrin solubility enhancers, such as a sulfoalkyl ether cyclodextrin (SAE-CD), especially SBE7-β-CD. In some embodiments, corticosteroid solutions of the invention demonstrate less than 10% loss of corticosteroid potency after 24 months under normal conditions (25° C. and 60% relative humidity). In some embodiments, budesonide solutions of the invention demonstrate less than 10% loss of budesonide potency after 24 months under normal conditions (25° C. and 60% relative humidity).

The foregoing and other needs are further met by embodiments of the invention, which provide a corticosteroid mixture which, upon exposing the corticosteroid mixture to normal or accelerated conditions (e.g.30° C., 40° C. or 60° C.) for a period of 3 or more months demonstrates about 0.1% to about 5%, about 0.2% to about 4%, about 0.5% to about 3%, about 0.7% to about 2%, about 0.8% to about 2% or about 1 to about 2%, less than about 10%, less than about 7.5% less than about 5%, less than about 4%, less than about 3%, less than about 2.5%, less than about 2.2% or about 2% or less degradation. In some preferred embodiments, the mixture is a corticosteroid solution, which optionally comprises one or more additional ingredients. Optional additional ingredients include solubility enhancers, especially cyclodextrin solubility enhancers, such as a sulfoalkyl ether cyclodextrin (SAE-CD), especially SBE7-β-CD. In some embodiments, corticosteroid solutions of the invention demonstrate less than 10% loss of corticosteroid potency after 24 months under normal conditions (25° C. and 60% relative humidity). In some embodiments, budesonide solutions of the invention demonstrate less than 10% loss of budesonide potency after 24 months under normal conditions (25° C. and 60% relative humidity).

The foregoing and other needs are further met by embodiments of the invention, which provide a corticosteroid mixture which, upon exposing the corticosteroid mixture to normal or accelerated conditions (e.g. 30° C., 40° C. or 60° C.) for a period of 6 months or more demonstrates about 0.1% to about 5%, about 0.2% to about 4%, about 0.5% to about 3%, about 0.7% to about 2%, about 0.8% to about 2% or about 1 to about 2%, less than about 10%, less than about 7.5% less than about 5%, less than about 4%, less than about 3%, less than about 2.5%, less than about 2.2% or about 2% or less degradation. In some preferred embodiments, the mixture is a corticosteroid solution, which optionally comprises one or more additional ingredients. Optional additional ingredients include solubility enhancers, especially cyclodextrin solubility enhancers, such as a sulfoalkyl ether cyclodextrin (SAE-CD), especially SBE7-β-CD. In some embodiments, corticosteroid solutions of the invention demonstrate less than 10% loss of corticosteroid potency after 24 months under normal conditions (25° C. and 60% relative humidity). In some embodiments, budesonide solutions of the invention demonstrate less than 10% loss of budesonide potency after 24 months under normal conditions (25° C. and 60% relative humidity).

The foregoing and other needs are further met by embodiments of the invention, which provide a corticosteroid mixture which, upon exposing the corticosteroid mixture to normal or accelerated conditions (e.g. 30° C., 40° C. or 60° C.) for a period of 12 months or more demonstrates about 0.1% to about 5%, about 0.2% to about 4%, about 0.5% to about 3%, about 0.7% to about 2%, about 0.8% to about 2% or about 1 to about 2%, less than about 10%, less than about 7.5% less than about 5%, less than about 4%, less than about 3%, less than about 2.5%, less than about 2.2% or about 2% or less degradation. In some preferred embodiments, the mixture is a corticosteroid solution, which optionally comprises one or more additional ingredients. Optional additional ingredients include solubility enhancers, especially cyclodextrin solubility enhancers, such as a sulfoalkyl ether cyclodextrin (SAE-CD), especially SBE7-β-CD. In some embodiments, corticosteroid solutions of the invention demonstrate less than 10% loss of corticosteroid potency after 24 months under normal conditions (25° C. and 60% relative humidity). In some embodiments, budesonide solutions of the invention demonstrate less than 10% loss of budesonide potency after 24 months under normal conditions (25° C. and 60% relative humidity).

The foregoing and other needs are further met by embodiments of the invention, which provide a corticosteroid mixture which, upon exposing the corticosteroid mixture to normal or accelerated conditions (e.g. 30° C., 40° C. or 60° C.) for period of 24 months or more demonstrates about 0.1% to about 5%, about 0.2% to about 4%, about 0.5% to about 3%, about 0.7% to about 2%, about 0.8% to about 2% or about 1 to about 2%, less than about 10%, less than about 7.5% less than about 5%, less than about 4%, less than about 3%, less than about 2.5%, less than about 2.2% or about 2% or less degradation. In some preferred embodiments, the mixture is a corticosteroid solution, which optionally comprises one or more additional ingredients. Optional additional ingredients include solubility enhancers, especially cyclodextrin solubility enhancers, such as a sulfoalkyl ether cyclodextrin (SAE-CD), especially SBE7-β-CD. In some embodiments, corticosteroid solutions of the invention demonstrate less than 10% loss of corticosteroid potency after 24 months under normal conditions (25° C. and 60% relative humidity). In some embodiments, budesonide solutions of the invention demonstrate less than 10% loss of budesonide potency after 24 months under normal conditions (25° C. and 60% relative humidity).

### BRIEF DESCRIPTION OF THE DRAWINGS

The novel features of the invention are set forth with particularity in the appended claims. A better understanding of the features and advantages of certain embodiments of the present invention will be obtained by reference to the following detailed description that sets forth illustrative embodiments, in which the principles of the invention are utilized, and the accompanying drawings of which:

FIG. 1 is a flow diagram illustrating an embodiment of a budesonide solution manufacturing process according to the present invention.

### INCORPORATION BY REFERENCE

All publications and patent applications mentioned in this specification are herein incorporated by reference to the same extent as if each individual publication or patent application was specifically and individually indicated to be incorporated by reference. In particular, the following WIPO Published Patent Applications, each of which designates the United States, are noted and are specifically incorporated herein in their entireties: WO 2005/065649, WO 2005/065435 and WO 2005/065651.

### DETAILED DESCRIPTION OF THE INVENTION

The present invention provides a process of making a stabilized corticosteroid mixture, especially a stabilized corticosteroid mixture, such as a corticosteroid solution, and most especially a stabilized mixture of budesonide, such as a stabilized budesonide solution. The invention comprises mixing corticosteroid, water and other ingredients, such as a solubility enhancer, pH adjusting agents, anti-oxidants, preservatives, and agents for adjusting tonicity of the solution, under conditions wherein the partial pressure of oxygen has been reduced in the mixing vessel—so-called oxygen-depleted conditions. Such oxygen-depleted conditions may be obtained by sparging solvent water with an inert gas, such as nitrogen (N₂) or argon (Ar) gas to drive off oxygen (O₂) gas from the solvent, maintaining the mixture under an inert gas mixture during mixing, maintaining the mixture under a reduced oxygen atmosphere, applying a vacuum to the mixing apparatus before, during and/or after mixing, and/or combinations of the foregoing. In addition, the method may include maintaining the mixture under oxygen-depleted conditions after the solution has been filtered to remove biological contaminants (e.g. through a 0.1-0.5 μm pore size filter, especially a 0.1 to 0.22 βm pore diameter filter such as a Millipore CVGL71TP3 0.22 μm filter) and/or during and after dispensing of the mixture into unit doses. See copending application Ser. No. ______, filed Feb. 15, 2007, entitled “Methods of Manufacturing Corticosteroid Solutions,” Attorney Docket Number 31622-718/201, which is incorporated herein by reference in its entirety. It is considered that maintaining the mixture under oxygen-depleted conditions during mixing provides superior stability of the mixture over time, as compared to purging of the mixture (after it has been terminally sterilized) alone.

In some embodiments, the invention provides a process of preparing a corticosteroid mixture, comprising mixing ingredients of the corticosteroid mixture in a mixing vessel under oxygen-depleted conditions to produce the corticosteroid mixture, wherein the ingredients include as starting materials corticosteroid and water. In some embodiments, the corticosteroid mixture is a budesonide mixture, and in some preferred embodiments, budesonide solution in water. In some embodiments, the invention further comprises storing the corticosteroid mixture in a holding tank for a storage period. The storage period may be varied, but in some preferred embodiments (e.g. where the corticosteroid is budesonide) the storage period should be such as to accommodate in process testing (e.g. potency testing, detection of impurities, and/or other testing known to those skilled in the pharmaceutical arts. In some preferred embodiments, the contents of the holding tank are under oxygen depleted conditions. In some embodiments, the corticosteroid mixture is then dispensed into pharmaceutically acceptable containers (e.g. bottles, ampoules, vials, etc.) In some preferred embodiments, the corticosteroid mixture is dispensed into pharmaceutically acceptable containers under oxygen-depleted conditions. In some embodiments, the pharmaceutically acceptable containers are then placed in pouches, which may be sealed to exclude ambient oxygen, sunlight, contaminants and/or tampering. In some preferred embodiments, the packaging of the pharmaceutically acceptable containers in pouches is carried out under oxygen-depleted conditions. In some embodiments, the corticosteroid mixture is a solution. In some embodiments, the corticosteroid mixture further comprises a solubility enhancer, such as a sulfoalkyl ether cyclodextrin (SAE-CD), e.g. SBE7-μ-CD. In some preferred embodiments, the corticosteroid is budesonide. In some alternative embodiments, the corticosteroid solution further comprises an additional active pharmaceutical ingredient, such as a short acting μ₂ agonist, preferably albuterol. The oxygen-depleted conditions may include, where applicable, one or more of the following procedures: sparging the water (e.g. water-for-injection; “WFI”), the corticosteroid mixture or both with inert gas (e.g. during mixing); applying inert gas over the water (e.g. before mixing), the mixture (e.g. during and/or after mixing) or both; or applying a vacuum to the water (e.g. prior to mixing), the mixture (e.g. during and/or after mixing) or both. In some embodiments, the inert gas is selected from nitrogen gas (N₂), argon gas (Ar) and mixtures thereof, with nitrogen gas being currently preferred. The invention further provides a corticosteroid mixture (especially a budesonide solution) prepared by the foregoing methodology.

The invention further provides a corticosteroid mixture, which loses no more than about 2% of corticosteroid potency after exposing the corticosteroid mixture to accelerated conditions of 40° C. and 75% relative humidity for a stability testing period of at least about 3 months, at least about 6 months, at least about 9 months or at least about 12 months. In this application, potency is measured by assaying representative containers (samples) of corticosteroid mixture at the start of stability testing (t₀) and at one or more predetermined time points, such as 3, 6, 9 and/or 12 months. The concentration of the corticosteroid in each sample is determined by a suitable detection method at each time point and the potency of corticosteroid at each time point (Cₜ; t=time point) is determined by known methods (e.g. averaging a plurality of samples from the same batch). The percent potency is then determined by applying the formula: Pₜ=100%, (Cₜ/C₀), wherein Pₜ is the potency (expressed in %) of corticosteroid remaining in the sample at time t, Cₜ is the concentration of corticosteroid (expressed in units such as μg/ml) in the mixture at time t, and C₀ is the concentration of corticosteroid (same units as Cₜ) in the mixture at the start of stability testing (by definition, t₀). By definition, P₀ is 100%. In some preferred embodiments, the mixture is a solution. In some preferred embodiments, the corticosteroid mixture further comprises a solubility enhancer, such as sulfoalkyl ether cyclodextrin (SAE-CD), e.g. SBE7-β-CD. In some preferred embodiments, the corticosteroid is budesonide. In some preferred embodiments, the mixture further comprises an additional active pharmaceutical ingredient, such as a short acting β₂ agonist, preferably albuterol. In some embodiments, the mixture is produced by a process comprising mixing ingredients of the corticosteroid mixture in a mixing vessel under oxygen-depleted conditions to produce the corticosteroid mixture, wherein the ingredients include as starting materials corticosteroid and water. In some embodiments, the process further comprises storing the corticosteroid mixture in a holding tank. In some preferred embodiments, the storing the mixture in the holding tank under oxygen-depleted conditions. In some embodiments, the process further comprises dispensing the corticosteroid mixture into pharmaceutically acceptable containers. In some preferred embodiments, the corticosteroid mixture is dispensed into pharmaceutically acceptable containers under oxygen-depleted conditions. In some embodiments, the pharmaceutically acceptable containers are further packaged in pharmaceutically acceptable pouches. In some embodiments, the packaging of the pharmaceutically acceptable containers in pouches is carried out under oxygen-depleted conditions.

The invention leads to enhanced stability of the corticosteroid compositions. In some embodiments, the invention provides less than 10% loss of corticosteroid potency up to 3, 6, 9 and 12 months under 5° C. conditions. The invention further provides less than 10% loss of corticosteroid potency up to 3, 6, 9 and 12 months under 25° C., 60 % relative humidity conditions. The invention also provides less than 10% loss of corticosteroid potency up to 3, 6, 9 and 12 months under 35° C., 65% relative humidity. Moreover, the invention provides less than 10% loss of corticosteroid potency up to 3, 6, 9 and 12 months under 40° C., 75% relative humidity. In some embodiments, the invention provides less than 5% loss of corticosteroid potency up to 3, 6, 9 and 12 months under 5° C. conditions. The invention further provides less than 5% loss of corticosteroid potency up to 3, 6, 9 and 12 months under 25° C., 60% relative humidity conditions. The invention also provides less than 5% loss of corticosteroid potency up to 3, 6, 9 and 12 months under 35° C., 65% relative humidity. Moreover, the invention provides less than 5% loss of corticosteroid potency up to 3, 6, 9 and 12 months under 40° C. 75% relative humidity. In some particular embodiments, the invention provides less than 3% loss of corticosteroid potency up to 3, 6, 9 and 12 months under 5° C. conditions. The invention further provides less than 3% loss of corticosteroid potency up to 3, 6, 9 and 12 months under 25° C., 60% relative humidity conditions. The invention also provides less than 3% loss of corticosteroid potency up to 3, 6, 9 and 12 months under 35° C., 65% relative humidity. Moreover, the invention provides less than 3% loss of corticosteroid potency up to 3, 6, 9 and 12 months under 40° C., 75% relative humidity. In some preferred embodiments, the invention provides less than about 2% loss of corticosteroid potency up to 3, 6, 9 and 12 months under 5° C. conditions. The invention further provides less than about 2% loss of corticosteroid potency up to 3, 6, 9 and 12 months under 25° C., 60% relative humidity conditions. The invention also provides less than about 2% loss of corticosteroid potency up to 3, 6, 9 and 12 months under 35° C., 65% relative humidity. Moreover, the invention provides less than about 2% loss of corticosteroid potency up to 3, 6, 9 and 12 months under 40° C., 75% relative humidity. Thus, the process of the invention, which comprises performing at least part of the mixing of corticosteroid and water under oxygen-depleted conditions (e.g. under an inert gas, such as nitrogen, under vacuum or both) provides for enhanced stability of the resulting corticosteroid solution. In some embodiments, corticosteroid solutions of the invention demonstrate less than 10% loss of corticosteroid potency after 24 months under normal conditions (25° C. and 60% relative humidity). In some embodiments, budesonide solutions of the invention demonstrate less than 10% loss of budesonide potency after 24 months under normal conditions (25° C. and 60% relative humidity).

The invention leads to enhanced stability of the budesonide compositions. In some embodiments, the invention provides less than 10% loss of budesonide potency up to 3, 6, 9 and 12 months under 5° C. conditions. The invention further provides less than 10% loss of budesonide potency up to 3, 6, 9, 12, 18 and 24 months under 25° C., 60% relative humidity conditions. The invention also provides less than 10% loss of budesonide potency up to 3, 6, 9 and 12 months under 35° C., 65% relative humidity. Moreover, the invention provides less than 10% loss of budesonide potency up to 3, 6, 9 and 12 months under 40° C., 75% relative humidity. In some embodiments, the invention provides less than 5% loss of budesonide potency up to 3, 6, 9 and 12 months under 5° C. conditions. The invention further provides less than 5% loss of budesonide potency up to 3, 6, 9 and 12 months under 25° C., 60% relative humidity conditions. The invention also provides less than 5% loss of budesonide potency up to 3, 6, 9 and 12 months under 35° C., 65% relative humidity. Moreover, the invention provides less than 5% loss of budesonide potency up to 3, 6, 9 and 12 months under 40° C., 75% relative humidity. In some particular embodiments, the invention provides less than 3% loss of budesonide potency up to 3, 6, 9 and 12 months under 5° C. conditions. The invention further provides less than 3% loss of budesonide potency up to 3, 6, 9 and 12 months under 25° C., 60% relative humidity conditions. The invention also provides less than 3% loss of budesonide potency up to 3, 6, 9 and 12 months under 35° C., 65% relative humidity. Moreover, the invention provides less than 3% loss of budesonide potency up to 3, 6, 9 and 12 months under 40° C., 75% relative humidity. In some preferred embodiments, the invention provides less than about 2% loss of budesonide potency up to 3, 6, 9 and 12 months under 5° C. conditions. The invention further provides less than about 2% loss of budesonide potency up to 3, 6, 9 and 12 months under 25° C., 60% relative humidity conditions. The invention also provides less than about 2% loss of budesonide potency up to 3, 6, 9 and 12 months under 35° C., 65% relative humidity. Moreover, the invention provides less than about 2% loss of budesonide potency up to 3, 6, 9 and 12 months under 40° C., 75% relative humidity. Thus, the process of the invention, which comprises performing at least part of the mixing of budesonide and water under oxygen-depleted conditions (e.g. under an inert gas, such as nitrogen, under vacuum or both) provides for enhanced stability of the resulting budesonide solution. In some embodiments, corticosteroid solutions of the invention demonstrate less than 10% loss of corticosteroid potency after 24 months under normal conditions (25° C. and 60% relative humidity). In some embodiments, budesonide solutions of the invention demonstrate less than 10% loss of budesonide potency after 24 months under normal conditions (25° C. and 60% relative humidity).

In some embodiments, the invention provides a process of preparing a corticosteroid mixture, comprising mixing ingredients of the corticosteroid mixture in a mixing vessel under oxygen-depleted conditions. The process results in a corticosteroid mixture having increased stability. In general, the mixture comprises corticosteroid and water. In preferred embodiments, the corticosteroid mixture is a solution, although it is considered that manufacture of corticosteroid suspensions under oxygen-depleted conditions will result in improved stability characteristics for the resulting suspension. Thus, in preferred embodiments, the mixture includes a solubility enhancer, which acts to increase the solubility of the corticosteroid in water. In especially preferred embodiments, the mixture includes sufficient solubility enhancer of such character as to solubilize substantially all the corticosteroid, thereby rendering a corticosteroid solution. Especially suitable solubility enhancers are set forth in more detail below; however preferred solubility enhancers belong to the family of solubility enhancers known as sulfoalkyl ether cyclodextrin (SAE-CD); and especially preferred SAE-CD compounds are those belonging to the sub-class of SBE-β-CD compounds, especially SBE7-β-CD. It is considered that the process is generally applicable to many corticosteroids, such as those set forth in more detail below. However, a preferred corticosteroid is budesonide, which heretofore has proven to be especially difficult to prepare in stable solutions. Thus, a preferred embodiment of the corticosteroid solution is a budesonide solution comprising SBE7-β-CD, water and optionally such other ingredients necessary to adjust and/or maintain the pH and tonicity of the solution. Other optional solubility enhancers include polysorbate 80. In some embodiments, the corticosteroid solution may comprise an additional active pharmaceutical ingredient. Suitable additional active pharmaceutical ingredients are those that cooperate with the corticosteroid active ingredient in the treatment of one or more conditions in the lung. Such additional active ingredients are known and disclosed in the art. Preferred additionally active ingredients include water soluble active ingredients, especially water soluble β₂ agonists, such as the short acting β₂ agonists, of which albuterol is a preferred embodiment with respect to the present invention. However, other active ingredients, as discussed in more detail below, can be substituted for or included with albuterol in the corticosteroid compositions of the invention.

In general, it is considered desirable to maintain the mixture under oxygen-depleted conditions during the duration of the mixing process. The term “oxygen-depleted” means a partial pressure of oxygen that is less than would be found under the same conditions without intervention to lower the partial pressure. The partial pressure of oxygen may be lowered e.g. by applying a vacuum to the mixture, which will draw off oxygen from the mixture and the overlying gas, or by applying a positive pressure of an inert gas such as N₂ or Ar, thereby causing oxygen to be displaced from the mixture by the inert gas. In some cases, a combination of methods may be used to achieve the desired result of reducing oxygen partial pressure over the mixture. For example, the solvent water may first be sparged with inert gas (either before or after it is charged into the mixing vessel); then the mixture may be subjected to inert gas overpressure during the mixing process; then the mixture may be discharged from the mixing vessel into a holding tank where it is overlayed with an inert gas. In other embodiments, the solvent water may first be sparged with inert gas (either before or after it is charged into the mixing vessel); then the mixture may be subjected to one or more cycles of vacuum followed by inert gas overpressure during the mixing process; then the mixture may be discharged from the mixing vessel into a holding tank where it is overlayed with an inert gas. Typical vacuum-inert gas overpressure cycles include a 1-10 minute (about 5 minute preferred) vacuum step followed by inert gas overpressure of about 1000 to about 3000, about 1000 to about 2500 mbar or about 1000 to about 1500 mbar (about 1200 mbar of N₂ preferred). The process may include from 1 to about 10, about 1 to 5, 1 to 3, and most particularly 2 such cycles.

Thus, the invention provides a method of preparing a corticosteroid mixture comprising water and corticosteroid, wherein the oxygen-depleted conditions include sparging the water with an inert gas prior to mixing. As mentioned above, certain embodiments of such conditions may also comprise additional conditions, such as inert gas overpressure applied during the mixing process, vacuum applied during the mixing process, at least one cycle of vacuum and inert gas overpressure during the mixing process, etc. In addition, the process can include discharging the mixture into a holding apparatus and overpressurizing the holding apparatus with about 1000 to about 3000 mbar or more of inert gas (about 2000 mbar preferred). Inert gases that may be used include nitrogen and argon gas, with nitrogen being preferred. Also as mentioned above, a preferred mixture comprises a solubility enhancer, such as SBE7-β-CD, and an especially preferred mixture comprises budesonide as the corticosteroid. The invention further provides a product mixture produced by such process, wherein the product mixture has enhanced stability as compared to a mixture of similar composition that is not mixed under oxygen-depleted conditions, such as a mixture that is mixed under normal oxygen partial pressure and is only terminally purged with nitrogen.

Thus, the invention provides a method of preparing a corticosteroid mixture comprising water and corticosteroid, wherein the oxygen-depleted conditions include, prior to combining the ingredients, purging all apparatuses used during said mixing with an inert gas. As mentioned above, certain embodiments of certain embodiments of the invention may also include one or more, and preferably two or more of the following: sparging the solvent water with an inert gas prior to mixing, applying inert gas overpressure applied during the mixing process, applying vacuum during the mixing process, applying at least one cycle of vacuum and inert gas overpressure during the mixing process, etc. In addition, the process can include discharging the mixture into a holding apparatus and overpressurizing the holding apparatus with about 1000 to about 3000 mbar of inert gas (about 2000 mbar preferred). Inert gases that may be used in the various steps of this process include nitrogen and argon gas, with nitrogen being preferred. Also as mentioned above, a preferred mixture comprises a solubility enhancer, such as SBE7-β-CD, and an especially preferred mixture comprises budesonide as the corticosteroid. The invention further provides a product mixture produced by such process, wherein the product mixture has enhanced stability as compared to a mixture of similar composition that is not mixed under oxygen-depleted conditions, such as a mixture that is mixed under normal oxygen partial pressure and is only terminally purged with nitrogen. Thus the invention provides a process of preparing a corticosteroid mixture which, upon exposing the corticosteroid solution to normal patient storage conditions for a period of about 1 week to about 24 months or more demonstrates about 0.1% to about 5%, about 0.2% to about 4%, about 0.5% to about 3%, about 0.7% to about 2%, about 0.8% to about 2% or about 1 to about 2%, less than about 10%, less than about 7.5% less than about 5%, less than about 4%, less than about 3%, less than about 2.5%, less than about 2.2% or about 2% or less degradation.

Thus, the invention provides a method of preparing a corticosteroid mixture comprising water and corticosteroid, wherein the oxygen-depleted conditions include maintaining all equipment and ingredients under an inert gas atmosphere during mixing. As mentioned above, certain embodiments of the invention may also include (and in preferred embodiments will include) one or more, and preferably two or more of the following: prior to combining the ingredients, purging all apparatuses used during said mixing with an inert gas, sparging the solvent water with an inert gas prior to mixing, applying inert gas overpressure applied during the mixing process, applying vacuum during the mixing process, applying at least one cycle of vacuum and inert gas overpressure during the mixing process, etc. In addition, the process can include discharging the mixture into a holding apparatus and overpressurizing the holding apparatus with about 1000 to about 3000 mbar of inert gas (about 2 bar preferred). Inert gases that may be used in the various steps of this process include nitrogen and argon gas, with nitrogen being preferred. Also as mentioned above, a preferred mixture comprises a solubility enhancer, such as SBE7-β-CD, and an especially preferred mixture comprises budesonide as the corticosteroid. The invention further provides a product mixture produced by such process, wherein the product mixture has enhanced stability as compared to a mixture of similar composition that is not mixed under oxygen-depleted conditions, such as a mixture that is mixed under normal oxygen partial pressure and is only terminally purged with nitrogen. Thus the invention provides a process of preparing a corticosteroid mixture which, upon exposing the corticosteroid solution to normal patient storage conditions for a period of about 1 week to about 24 months or more demonstrates about 0.1% to about 5%, about 0.2% to about 4%, about 0.5% to about 3%, about 0.7% to about 2%, about 0.8% to about 2% or about 1 to about 2%, less than about 10%, less than about 7.5% less than about 5%, less than about 4%, less than about 3%, less than about 2.5%, less than about 2.2% or about 2% or less degradation.

In the context of the present invention, “normal patient storage conditions” or “normal conditions” means storage at 25° C. and 60% relative humidity (“25/60”). Normal patient storage conditions are intended to simulate the conditions under which a normal patient would usually store the drug over an extended period of time, e.g. several weeks to at least about 24 months. The term “accelerated conditions,” unless otherwise specified, means storage at 40° C. and 75% relative humidity (“40/75”). Other storage conditions will be specified by reference to the temperature and relative humidity.

Thus, the invention further provides a method of preparing a corticosteroid mixture comprising water and corticosteroid, wherein the oxygen-depleted conditions include further include purging pharmaceutically acceptable containers to be filled with the corticosteroid (e.g. budesonide) solution with an inert gas. As mentioned above, certain embodiments of the invention may also include (and in preferred embodiments will include) one or more, and preferably two or more of the following: maintaining all equipment and ingredients under an inert gas atmosphere during mixing, prior to combining the ingredients, purging all apparatuses used during said mixing with an inert gas, sparging the solvent water with an inert gas prior to mixing, applying inert gas overpressure applied during the mixing process, applying vacuum during the mixing process, applying at least one cycle of vacuum and inert gas overpressure during the mixing process, etc. In addition, the process can include discharging the mixture into a holding apparatus and overpressurizing the holding apparatus with about 1000 mbar to about 3000 mbar of inert gas (about 2000 bar preferred). Inert gases that may be used in the various steps of this process include nitrogen and argon gas, with nitrogen being preferred. Also as mentioned above, a preferred mixture comprises a solubility enhancer, such as SBE7-β-CD, and an especially preferred mixture comprises budesonide as the corticosteroid. The invention further provides a product mixture produced by such process, wherein the product mixture has enhanced stability as compared to a mixture of similar composition that is not mixed under oxygen-depleted conditions, such as a mixture that is mixed under normal oxygen partial pressure and is only terminally purged with nitrogen. Thus the invention provides a process of preparing a corticosteroid mixture which, upon exposing the corticosteroid solution to normal patient storage conditions for a period of about 1 week to about 24 months or more demonstrates about 0.1% to about 5%, about 0.2% to about 4%, about 0.5% to about 3%, about 0.7% to about 2%, about 0.8% to about 2% or about 1 to about 2%, less than about 10%, less than about 7.5% less than about 5%, less than about 4%, less than about 3%, less than about 2.5%, less than about 2.2% or about 2% or less degradation.

Thus, the invention further provides a method of preparing a corticosteroid mixture comprising water and corticosteroid, wherein the oxygen-depleted conditions include maintaining the mixing vessel under vacuum during at least part of the mixing process. As mentioned above, certain embodiments of the invention may also include (and in preferred embodiments will include) one or more, and preferably two or more of the following: purging pharmaceutically acceptable containers to be filled with the budesonide solution with an inert gas; maintaining all equipment and ingredients under an inert gas atmosphere during mixing; prior to combining the ingredients; purging all apparatuses used during said mixing with an inert gas; sparging the solvent water with an inert gas prior to mnixing; applying inert gas overpressure applied during the mixing process, applying vacuum during the mixing process. In some preferred embodiments, vacuum and inert gas overpressure are applied as at least one cycle, and preferably at least two cycles of vacuum followed by inert gas overpressure or the converse. In addition, the process can include discharging the mixture into a holding apparatus and overpressurizing the holding apparatus with about 1000 to about 3000 mbar of inert gas (about 2000 mbar preferred). Inert gases that may be used in the various steps of this process include nitrogen and argon gas, with nitrogen being preferred. Also as mentioned above, a preferred mixture comprises a solubility enhancer, such as SBE7-β-CD, and an especially preferred mixture comprises budesonide as the corticosteroid. The invention further provides a product mixture produced by such process, wherein the product mixture has enhanced stability as compared to a mixture of similar composition that is not mixed under oxygen-depleted conditions, such as a mixture that is mixed under normal oxygen partial pressure and is only terminally purged with nitrogen. Thus the invention provides a process of preparing a corticosteroid mixture which, upon exposing the corticosteroid solution to normal patient storage conditions for a period of about 1 week to about 24 months or more demonstrates about 0.1% to about 5%, about 0.2% to about 4%, about 0.5% to about 3%, about 0.7% to about 2%, about 0.8% to about 2% or about 1 to about 2%, less than about 10%, less than about 7.5% less than about 5%, less than about 4%, less than about 3%, less than about 2.5%, less than about 2.2% or about 2% or less degradation.

Furthermore, the invention provides a corticosteroid mixture which, after exposing the corticosteroid mixture to accelerated conditions of 40° C. and 75% relative humidity for 3 months, demonstrates no more than about 2% degradation of the corticosteroid in the mixture. In preferred embodiments, the mixture is in the form of a solution, although it is considered that the same general methodology will improve the stability characteristics of corticosteroid suspensions as well. Corticosteroids in general, and budesonide specifically, have low solubility in water. Hence, in the preferred corticosteroid solutions a solubility enhancer is included to enhance the solubility of the corticosteroid. In particular solutions, the preferred corticosteroid is budesonide. Solubility enhancers are set forth below; however a preferred class of solubility enhancers includes the sulfoalkyl ether cyclodextrin (SAE-CD), especially a member of the class of SBE-β-CD compounds, and preferably SBE7-β-CD, which is also known by its trade name Captisol®. Thus a preferred embodiment of the mixture of the invention comprises budesonide, SBE7-β-CD, water and optionally such inert ingredients as are necessary to prepare a pharmaceutically acceptable solution, such as pH and tonicity adjusters. In some embodiments, the corticosteroid mixture includes an additional active ingredient. Preferred active ingredients include those which cooperate with the corticosteroid in the treatment of one or more disorders of the lungs, such as bronchial spasm, bronchial inflammation, excessive phlegm viscosity, etc. In particular embodiments, it is considered preferable to use an additional active ingredient that is soluble in water. Suitable active ingredients are discussed in detail below; however a preferred class of additional active ingredients is the short-acting β₂ agonists, such as albuterol, which is preferred. Thus the invention provides a process of preparing a corticosteroid mixture which, upon exposing the filled and pouched corticosteroid solution to normal patient storage conditions for a period of about 1 week to about 24 months or more demonstrates about 0.1% to about 5%, about 0.2% to about 4%, about 0.5% to about 3%, about 0.7% to about 2%, about 0.8% to about 2% or about 1 to about 2%, less than about 10%, less than about 7.5% less than about 5%, less than about 4%, less than about 3%, less than about 2.5%, less than about 2.2% or about 2% or less degradation.

The term “solubility enhancer” means a pharmaceutically inert ingredient that enhances the solubility of corticosteroid in water. In some embodiments, the solubility enhancer is selected from the group consisting of propylene glycol, non-ionic surfactants, tyloxapol, polysorbate 80, vitamin E-TPGS, macrogol-15-hydroxystearate, phospholipids, lecithin, purified and/or enriched lecithin, phosphatidylcholine fractions extracted from lecithin, dimyristoyl phosphatidylcholine (DMPC), dipalmitoyl phosphatidylcholine (DPPC), distearoyl phosphatidylcholine (DSPC), cyclodextrins and derivatives thereof, SAE-CD derivatives, SBE-α-CD, SBE-β-CD, SBE-γ-CD, dimethyl β-CD, hydroxypropyl-β-cyclodextrin, 2-HP-β-CD, hydroxyethyl-β-cyclodextrin, hydroxypropyl-λ-cyclodextrin, hydroxyethyl-γ-cyclodextrin, dihydroxypropyl-β-cyclodextrin, glucosyl-α-cyclodextrin, glucosyl-β-cyclodextrin, diglucosyl-β-cyclodextrin, maltosyl-α-cyclodextrin, maltosyl-β-cyclodextrin, maltosyl-γ-cyclodextrin, maltotriosyl-β-cyclodextrin, maltotriosyl-γ-cyclodextrin, dimaltosyl-β-cyclodextrin, methyl-β-cyclodextrin, carboxyalkyl thioether derivatives, ORG 26054, ORG 25969, hydroxypropyl methylcellulose, hydroxypropylcellulose, polyvinylpyrrolidone, copolymers of vinyl acetate, vinyl pyrrolidone, sodium lauryl sulfate, dioctyl sodium sulfosuccinate, and combinations thereof. In particular embodiments, SAE-CD derivatives are preferred. In particularly preferred embodiments, the SAE-CD derivatives belonging to the group of SBE-β-CD derivatives are preferred. In specific embodiments, a particularly preferred solubility enhancer is SBE7-β-CD. In some embodiments, Polysorbate 80 is included in the formulation at concentrations of about 0.01% or less, especially about 0.005% or less, and more specifically about 0.001% or less; while in other embodiments it is preferred to substantially exclude Polysorbate 80 from the corticosteroid solution. In preferred embodiments, the corticosteroid solution contains a molar excess of SAE-CD derivative, especially SBE7-β-CD, with respect to the corticosteroid, especially budesonide.

In some embodiments of the invention, the corticosteroid mixture further comprises a solubility enhancer. The term “solubility enhancer” means a pharmaceutically inert ingredient that enhances the solubility of corticosteroid in water. In some embodiments, the solubility enhancer can have a concentration (w/v) ranging from about 0.001% to about 25%. In other embodiments, the solubility enhancer can have a concentration (w/v) ranging from about 0.01% to about 20%. In still other embodiments, the solubility enhancer can have a concentration (w/v) ranging from about 0.1% to about 15%. In yet other embodiments, the solubility enhancer can have a concentration (w/v) ranging from about 1% to about 10%. In a preferred embodiment, the solubility enhancer can have a concentration (w/v) ranging from about 1% to about 8% when the solubility enhancer is a cyclodextrin or cyclodextrin derivative.

A “solubility enhancer,” as used herein, includes one or more compounds which increase the solubility of corticosteroid in the aqueous phase of the corticosteroid mixture. In general the solubility enhancer increases the solubility of the corticosteroid in water without chemically changing the corticosteroid. In particular, the solubility enhancer increases the solubility of corticosteroid without substantially decreasing, and in some embodiments increasing, the activity of the corticosteroid.

Solubility enhancers are known in the art and are described in, e.g., U.S. Pat. Nos. 5,134,127, 5,145,684, 5,376,645, 6,241,969 and U.S. Pub. Appl. Nos. 2005/0244339 and 2005/0008707, each of which is specifically incorporated by reference herein. In addition, examples of suitable solubility enhancers are described below.

Solubility enhancers suitable for use in the present invention include, but are not limited to, propylene glycol, non-ionic surfactants, phospholipids, cyclodextrins and derivatives thereof, and surface modifiers and/or stabilizers.

Examples of non-ionic surfactants which appear to have a particularly good physiological compatibility for use in the present invention are tyloxapol, polysorbates including, but not limited to, polyoxyethylene (20) sorbitan monolaurate, polyoxyethylene (20) sorbitan monopalmitate, polyoxyethylene (20) sorbitan monostearate (available under the trade name Tweens 20-40-60, etc.), Polysorbate 80, Polyethylene glycol 400; sodium lauryl sulfate; sorbitan laurate, sorbitan palmitate, sorbitan stearate (available under the trade name Span 20-40-60 etc.), benzalkonium chloride, PPO-PEO block copolymers (Pluronics), Cremophor-EL, vitamin E-TPGS (e.g., d-alpha-tocopheryl-polyethyleneglycol-1000-succinate), Solutol-HS-15, oleic acid PEO esters, stearic acid PEO esters, Triton-X100, Nonidet P-40, and macrogol hydroxystearates such as macrogol-15-hydroxystearate.

In some embodiments, the non-ionic surfactants suitable for use in the present invention are formulated with the corticosteroid to form liposome preparations, micelles or mixed micelles. Methods for the preparation and characterization of liposomes and liposome preparations are known in the art. Often, multi-lamellar vesicles will form spontaneously when amphiphilic lipids are hydrated, whereas the formation of small uni-lamellar vesicles usually requires a process involving substantial energy input, such as ultrasonication or high pressure homogenization. Further methods for preparing and characterizing liposomes have been described, for example, by S. Vemuri et al. (Preparation and characterization of liposomes as therapeutic delivery systems: a review in Pharm Acta Helv. 1995, 70(2):95-111) and U.S. Pat. Nos. 5,019,394, 5,192,228, 5,882,679, 6,656,497 each of which is specifically incorporated by reference herein.

In some cases, for example, micelles or mixed micelles may be formed by the surfactants, in which poorly soluble active agents can be solubilized. In general, micelles are understood as substantially spherical structures formed by the spontaneous and dynamic association of amphiphilic molecules, such as surfactants. Mixed micelles are micelles composed of different types of amphiphilic molecules. In this context, both micelles and mixed micelles should not be understood as solid particles, as their structure, properties and behavior are much different from solids. The amphiphilic molecules which form the micelles usually associate temporarily. In a micellar solution, there is a dynamic exchange of molecules between the micelle-forming amphiphile and monomolecularly dispersed amphiphiles which are also present in the solution. The position of the drug molecules which are solubilized in such micelles or mixed micelles depends on the structure of these molecules as well as the surfactants used. For example, it is to be assumed that particularly non-polar molecules are localized mainly inside the colloidal structures, whereas polar substances are more likely to be found on the surface. In one embodiment of a micellar or mixed micellar solution, the average size of the micelles may be less than about 200 nm (as measured by photon correlation spectroscopy), such as from about 10 nm to about 100 nm. Particularly preferred are micelles with average diameters of about 10 to about 50 nm. Methods of producing micelles and mixed micelles are known in the art and described in, for example, U.S. Pat. Nos. 5,747,066 and 6,906,042, each of which is specifically incorporated by reference herein.

Phospholipids are defined as amphiphilic lipids which contain phosphorus. Phospholipids which are chemically derived from phosphatidic acid occur widely and are also commonly used for pharmaceutical purposes. This acid is a usually (doubly) acylated glycerol-3-phosphate in which the fatty acid residues may be of different length. The derivatives of phosphatidic acid include, for example, the phosphocholines or phosphatidylcholines, in which the phosphate group is additionally esterified with choline, furthermore phosphatidyl ethanolamines, phosphatidyl inositols, etc. Lecithins are natural mixtures of various phospholipids which usually have a high proportion of phosphatidyl cholines. Depending on the source of a particular lecithin and its method of extraction and/or enrichment, these mixtures may also comprise significant amounts of sterols, fatty acids, tryglycerides and other substances.

Additional phospholipids which are suitable for compositions according to the present invention on account of their physiological properties comprise, in particular, phospholipid mixtures which are extracted in the form of lecithin from natural sources such as soja beans (soy beans) or chickens egg yolk, preferably in hydrogenated form and/or freed from lysolecithins, as well as purified, enriched or partially synthetically prepared phopholipids, preferably with saturated fatty acid esters. Of the phospholipid mixtures, lecithin is particularly preferred. The enriched or partially synthetically prepared medium- to long-chain zwitterionic phospholipids are mainly free of unsaturations in the acyl chains and free of lysolecithins and peroxides. Examples for enriched or pure compounds are dimyristoyl phosphatidyl choline (DMPC), distearoyl phosphatidyl choline (DSPC) and dipalmitoyl phosphatidyl choline (DPPC). Of these, DMPC is currently more preferred. Alternatively, phospholipids with oleyl residues and phosphatidyl glycerol without choline residue are suitable for some embodiments and applications of the invention.

In some embodiments, the non-ionic surfactants and phospholipids suitable for use in the present invention are formulated with the corticosteroid to form colloidal structures. Colloidal solutions are mono-phasic systems wherein the colloidal material dispersed within the colloidal solution does not have the measurable physical properties usually associated with a solid material. Methods of producing colloidal dispersions are known in the art, for example as described in U.S. Pat. No. 6,653,319, which is specifically incorporated by reference herein.

Suitable cyclodextrins and derivatives for use in the present invention are described in the art, for example, Challa et al., AAPS PharmSciTech 6(2): E329-E357 (2005), U.S. Pat. Nos. 5,134,127, 5,376,645, 5,874,418, each of which is specifically incorporated by reference herein. In some embodiments, suitable cyclodextrins or cyclodextrin derivatives for use in the present invention include, but are not limited to, α-cyclodextrins, α-cyclodextrins, γ-cyclodextrins, SAE-CD derivatives (e.g., SBE-α-CD, SBE-β-CD (Captisol®), and SBE-γ-CD) (CyDex, Inc. Lenexa, Kans.), hydroxyethyl, hydroxypropyl (including 2-and 3-hydroxypropyl) and dihydroxypropyl ethers, their corresponding mixed ethers and further mixed ethers with methyl or ethyl groups, such as methylhydroxyethyl, ethyl-hydroxyethyl and ethyl-hydroxypropyl ethers of α-, β- and γ-cyclodextrin; and the maltosyl, glucosyl and maltotriosyl derivatives of α-, β-and γ-cyclodextrin, which may contain one or more sugar residues, e.g. glucosyl or diglucosyl, maltosyl or dimaltosyl, as well as various mixtures thereof, e.g. a mixture of maltosyl and dimaltosyl derivatives. Specific cyclodextrin derivatives for use herein include hydroxypropyl-β-cyclodextrin, hydroxyethyl-β-cyclodextrin, hydroxypropyl-γ-cyclodextrin, hydroxyethyl-γ-cyclodextrin, dihydroxypropyl-β-cyclodextrin, glucosyl-α-cyclodextrin, glucosyl-β-cyclodextrin, diglucosyl-β-cyclodextrin, maltosyl-α-cyclodextrin maltosyl-β-cyclodextrin, maltosyl-γ-cyclodextrin, maltotriosyl-β-cyclodextrin, maltotriosyl-γ-cyclodextrin, dimaltosyl-β-cyclodextrin, diethyl-β-cyclodextrin, glucosyl-α-cyclodextrin, glucosyl-β-cyclodextrin, diglucosyl-β-cyclodextrin, tri-O-methyl-β-cyclodextrin, tri-O-ethyl-β-cyclodextrin, tri-O-butyryl-β-cyclodextrin, tri-O-valeryl-β-cyclodextrin, and di-O-hexanoyl-β-cyclodextrin, as well as methyl-β-cyclodextrin, and mixtures thereof such as maltosyl-β-cyclodextrin/dimaltosyl-β-cyclodextrin. Procedures for preparing such cyclodextrin derivatives are well-known, for example, from U.S. Pat. No. 5,024,998, and references incorporated by reference therein. Other cyclodextrins suitable for use in the present invention include the carboxyalkyl thioether derivatives such as ORG 26054 and ORG 25969 by ORGANON (AKZO-NOBEL), hydroxybutenyl ether derivatives by EASTMAN, sulfoalkyl-hydroxyalkyl ether derivatives, sulfoalkyl-alkyl ether derivatives, and other derivatives, for example as described in U.S. Patent Application Nos. 2002/0128468, 2004/0106575, 2004/0109888, and 2004/0063663, or U.S. Pat. Nos. 6,610,671, 6,479,467, 6,660,804, or 6,509,323, each of which is specifically incorporated by reference herein.

Hydroxypropyl-β-cyclodextrin can be obtained from Research Diagnostics Inc. (Flanders, N.J.). Exemplary hydroxypropyl-β-cyclodextrin products include Encapsin® (degree of substitution ˜4) and Molecusol® (degree of substitution ˜8); however, embodiments including other degrees of substitution are also available and are within the scope of the present invention.

Dimethyl cyclodextrins are available from FLUKA Chemnie (Buchs, CH) or Wacker (Iowa). Other derivatized cyclodextrins suitable for use in the invention include water soluble derivatized cyclodextrins. Exemplary water-soluble derivatized cyclodextrins include carboxylated derivatives; sulfated derivatives; alkylated derivatives; hydroxyalkylated derivatives; methylated derivatives; and carboxy-β-cyclodextrins, e.g., succinyl-β-cyclodextrin (SCD). All of these materials can be made according to methods known in the art and/or are available commercially. Suitable derivatized cyclodextrins are disclosed in Modified Cyclodextrins: Scaffolds and Templates for Supramolecular Chemistry (Eds. Christopher J. Easton, Stephen F. Lincoln, Imperial College Press, London, UK, 1999).

Suitable surface modifiers for use in the present invention are described in the art, for example, U.S. Pat. Nos. 5,145,684, 5,510,118, 5,565,188, and 6,264,922, each of which is specifically incorporated by reference herein. Examples of surface modifiers and/or surface stabilizers suitable for use in the present invention include, but are not limited to, hydroxypropyl methylcellulose, hydroxypropylcellulose, polyvinylpyrrolidone, sodium lauryl sulfate, dioctylsulfosuccinate, gelatin, casein, lecithin (phosphatides), dextran, gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers (e.g., macrogol ethers such as cetomacrogol 1000), polyoxyethylene castor oil derivatives, polyoxyethylene sorbitan fatty acid esters (e.g., the commercially available Tweens™, e.g., Tween 20™ and Tween 80™ (ICI Specialty Chemicals)), polyethylene glycols (e.g., Carbowax 3550™ and 934™ (Union Carbide)), polyoxyethylene stearates, colloidal silicon dioxide, phosphates, carboxymethylcellulose calcium, carboxymethylcellulose sodium, methylcellulose, hydroxyethylcellulose, hydroxypropylmethylcellulose phthalate, noncrystalline cellulose, magnesium aluminum silicate, triethanolamine, polyvinyl alcohol (PVA), 4-(1,1,3,3-tetramethylbutyl)-phenol polymer with ethylene oxide and formaldehyde (also known as tyloxapol, superione, and triton), poloxamers (e.g., Pluronics F68™ and F108™, which are block copolymers of ethylene oxide and propylene oxide), poloxamines (e.g., Tetronic 908™, also known as Poloxamine 908™, which is a tetrafunctional block copolymer derived from sequential addition of propylene oxide and ethylene oxide to ethylenediamine (BASF Wyandotte Corporation, Parsippany, N.J.)), Tetronic 1508™ (T-1508) (BASF Wyandotte Corporation), Tritons X-200™, which is an alkyl aryl polyether sulfonate (Rohm and Haas), Crodestas F-100™, which is a mixture of sucrose stearate and sucrose distearate (Croda Inc.), p-isononylphenoxypoly-(glycidol), also known as Olin-10G™ or Surfactant 10™ (Olin Chemicals, Stamford, Conn.), Crodestas SL-40® (Croda, Inc.), and SA9OHCO, which is C₁₈H₃₇CH₂(-CON(CH₃)—CH₂(CHOH)₄(CH₂OH)₂ (Eastman Kodak Co.), decanoyl-N-methylglucamide, n-decyl-β-D-glucopyranoside, n-decyl-β-D-maltopyranoside, n-dodecylβ-D-glucopyranoside, n-dodecyl-β-D-maltoside, heptanoyl-N-methylglucamide, n-heptyl-β-D-glucopyranoside, n-heptyl-β-thioglucoside, n-hexyl-β-D-glucopyranoside, nonanoyl-N-methylglucamide, n-nonanoyl-β-D-glucopyranoside, octanoyl-N-methylglucamide, n-octyl-β-D-glucopyranoside, octylβ-D-thioglucopyranoside, PEG-phospholipid, PEG-cholesterol, PEG-cholesterol derivative, PEG-vitamin A, PEG-vitamin E, lysozyme, random copolymers of vinyl pyrrolidone and vinyl acetate, and the like. (e.g. hydroxypropyl methylcellulose, hydroxypropylcellulose, polyvinylpyrrolidone, copolymers of vinyl acetate, vinyl pyrrolidone, sodium lauryl sulfate and dioctyl sodium sulfosuccinate).

Other useful cationic stabilizers include, but are not limited to, cationic lipids, sulfonium, phosphonium, and quarternary ammonium compounds, such as stearyltrimethylammonium chloride, benzyl-di(2-chloroethyl)ethylammonium bromide, coconut trimethyl ammonium chloride or bromide, coconut methyl dihydroxyethyl ammonium chloride or bromide, decyl triethyl ammonium chloride, decyl dimethyl hydroxyethyl ammonium chloride or bromide, C₁₂₋₁₅ dimethyl hydroxyethyl arumonium chloride or bromide, coconut dimethyl hydroxyethyl ammonium chloride or bromide, myristyl trimethyl ammonium methyl sulphate, lauryl dimethyl benzyl ammonium chloride or bromide, lauryl dimethyl (ethenoxy)₄ ammonium chloride or bromide, N-alkyl (C₁₂₋₁₈) dimethylbenzyl ammonium chloride, N-alkyl (C₁₄₋₁₈)dimethyl-benzyl ammonium chloride, N-tetradecylidmethylbenzyl ammonium chloride monohydrate, dimethyl didecyl ammonium chloride, N-alkyl and (C₁₂₋₁₄) dimethyl 1-napthylmethyl ammonium chloride, trimethylammonium halide, alkyl-trimethylammonium salts and dialkyl-dimethylammonium salts, lauryl trimethyl ammonium chloride, ethoxylated alkyamidoalkyldialkylanmuonium salt and/or an ethoxylated trialkyl ammonium salt, dialkylbenzene dialkylammonium chloride, N-didecyldimethyl ammonium chloride, N-tetradecyldimethylbenzyl ammonium, chloride monohydrate, N-alkyl(C₁₂₋₁₄) dimethyl 1-naphthylmethyl ammonium chloride and dodecyldimethylbenzyl ammonium chloride, dialkyl benzenealkyl ammonium chloride, lauryl trimethyl ammonium chloride, alkylbenzyl methyl ammonium chloride, alkyl benzyl dimethyl ammonium bromide, C₁₂, C₁₅, C₁₇ trimethyl ammonium bromides, dodecylbenzyl triethyl ammonium chloride, poly-diallyldimethylammonium chloride (DADMAC), dimethyl ammonium chlorides, alkyldimethylammonium halogenides, tricetyl methyl ammonium chloride, decyltrimethylammonium bromide, dodecyltriethylammonium bromide, tetradecyltrimethylammonium bromide, methyl trioctylammonium chloride (ALIQUAT 336™), POLYQUAT 10™, tetrabutylammonium bromide, benzyl trimethylammonium bromide, choline esters (such as choline esters of fatty acids), benzalkonium chloride, stearalkonium chloride compounds (such as stearyltrimonium chloride and Di-stearyldimonium chloride), cetyl pyridinium bromide or chloride, halide salts of quatemized polyoxyethylalkylamines, Mirapol™ and ALKAQUAT™ (Alkaril Chemical Company), alkyl pyridinium salts, amines, such as alkylamines, dialkylamines, alkanolamines, polyethylenepolyamines, N,N-dialkylaminoalkyl acrylates, and vinyl pyridine, amine salts, such as lauryl amine acetate, stearyl amine acetate, alkylpyridinium salt, and alkylimidazolium salt, and amine oxides, imide azolinium salts, protonated quaternary acrylamides, methylated quaternary polymers, such as poly[diallyl dimethylanunonium chloride] and poly-[N-methyl vinyl pyridinium chloride], and cationic guar.

In addition to aqueous mixtures comprising a corticosteroid and a solubility enhancer, it is contemplated herein that aqueous mixtures formulated by methods which provide enhanced solubility are likewise suitable for use in the presently disclosed invention. Thus, in the context of the present invention, a “solubility enhancer” includes aqueous mixtures formulated by methods which provide enhanced solubility with or without a chemical agent acting as a solubility enhancer. Such methods include, e.g., the preparation of supercritical fluids. In accordance with such methods, corticosteroid compositions, such as budesonide, are fabricated into particles with narrow particle size distribution (usually less than 200 nanometers spread) with a mean particle hydrodynamic radius in the range of 50 nanometers to 700 nanometers. The nano-sized corticosteroid particles, such as budesonide particles, are fabricated using Supercritical Fluids (SCF) processes including Rapid Expansion of Supercritical Solutions (RESS), or Solution Enhanced Dispersion of Supercritical fluids (SEDS), as well as any other techniques involving supercritical fluids. The use of SCF processes to form particles is reviewed in Palakodaty, S., et al., Pharmaceutical Research 16:976-985 (1999) and described in Bandi et al., Eur. J. Pharm. Sci. 23:159-168 (2004), U.S. Pat. No. 6,576,264 and U.S. Patent Application No. 2003/0091513, each of which is specifically incorporated by reference herein. These methods permit the formation of micron and sub-micron sized particles with differing morphologies depending on the method and parameters selected. In addition, these nanoparticles can be fabricated by spray drying, lyophilization, volume exclusion, and any other conventional methods of particle reduction.

Specific solubility enhancers or compounds that may be mentioned within the scope of the invention include polysorbate 80 and SAE-CD derivatives, SBE-α-CD, SBE-β-CD, SBE-γ-CD and dimethyl β-CD, hydroxypropyl-β-cyclodextrin, 2-HP-β-CD. In particular embodiments, SAE-CD derivatives are preferred. In particularly preferred embodiments, the SAE-CD derivatives belonging to the group of SBE-β-CD derivatives are preferred. In specific embodiments, a particularly preferred solubility enhancer is SBE7-β-CD. In some embodiments, Polysorbate 80 is included in the formulation at concentrations of about 0.01% or less, especially about 0.005% or less, and more specifically about 0.001% or less; while in other embodiments it is preferred to substantially exclude Polysorbate 80 from the corticosteroid solution. In preferred embodiments, the corticosteroid solution contains a molar excess of SAE-CD derivative, especially SBE7-β-CD, with respect to the corticosteroid, especially budesonide.

The term corticosteroid is intended to have the full breadth understood by those of skill in the art. Particular corticosteroids contemplated within the scope of the invention are those that are not generally soluble in water to a degree suitable for pharmaceutical administration, and thus require the presence of a solubility enhancer to dissolve them in aqueous solution. Particular corticosteroids that may be mentioned in this regard include those set forth in WO 2005/065649, WO 2005/065435 and WO 2005/065651. See in particular page 46 of WO 2005/065651, which is incorporated herein by reference. The corticosteroids that may be substituted for budesonide include aldosterone, beclomethasone, betamethasone, ciclesonide, cloprednol, cortisone, cortivazol, deoxycortone, desonide, desoximetasone, dexamethasone, difluorocortolone, fluclorolone, flumethasone, flunisolide, flucinolone, fluocinonide, fluocortin butyl, fluocortisone, flurocortolone, fluorometholone, flurandrenolone, fluticasone, halcinonide, hydrocortisone, icomethasone, meprednisone, methylpredinsolone, mometasone, paramethasone, prednisolone, prednisone, rofleponide, RPR 106541, tixocortol, triamcinolone and their pharmaceutically active derivatives, including prodrugs and pharmaceutically acceptable salts. In some embodiments, the invention may include a combination of two or more of the corticosteroids from the foregoing list. In some embodiments, the invention includes a combination of budesonide with one or more corticosteroids from the foregoing list.

The concentration of corticosteroid in the corticosteroid composition may vary from about 1 μg/ml to about 2000 μg/ml, about 1 μg/ml to about 1000 μg/ml or about 1 to about 500 μg/ml, especially about 50 μg/ml to about 500 μg/ml, or about 100 to about 400 μg/ml. Particular values that may be mentioned are about 1, about 5 μg/ml, about 10 μg/ml, about 20 μg/ml, about 50 μg/ml, about 100 μg/ml and about 200 μg/ml and about 250 μml. In some preferred embodiments, the corticosteroid concentration is about 80 μg/ml, about 120 μg/ml, about 240 μg/ml or about 480 μg/ml.

In addition to corticosteroid, the corticosteroid solution may include other active ingredients, especially other water-soluble active ingredients. Particularly suitable active ingredients are those that act either in conjunction with, or synergistically with, the corticosteroid for the treatment of one or more symptoms of respiratory disease, such as bronchial spasm, inflammation of bronchia, etc. The corticosteroid thus may be compounded with one or more other drugs, such as β₂ adrenoreceptor agonists (such as albuterol), dopamine D₂ receptor antagonists, anticholinergic agents or topical anesthetics. Specific active ingredients are known in the art, and preferred embodiments are set forth on pages 48-49 of WO 2005/065651, which pages are expressly incorporated herein by reference in their entirety.

In some embodiments, other active ingredients, especially water soluble active ingredients are included in the corticosteroid solution. In some preferred embodiments, the corticosteroid solution includes a water soluble short acting β₂-agonist, such as albuterol. Thus, some preferred embodiments include budesonide, a molor excess (relative to budesonide) of a cyclodextrin solubility enhancer, such as SBE7-β-CD, and albuterol.

In some preferred embodiments, the corticosteroid solution is manufactured by mixing a mass of corticosteroid starting material with the other ingredients in a high sheer mixer for less than about 5, less than about 4, less than about 3 and in particular about 2 hours or less. Preferably, such mixing is conducted under nitrogen. In particular embodiments, the mixing is carried out in a high sheer mixer having a capacity of at least about 10 L, at least about 50 L, at least about 100 L, at least about 250 L or at least about 500 L. In some such preferred embodiments, the mixing is carried out with alternating cycles of vacuum and overlay with positive inert gas (such as N₂ or Ar) pressure. In some specific embodiments, after mixing the solution is stored under an inert gas overlay (N₂ or Ar) of at least about 100 mbar, at least about 200 mbar, at least about 500 mbar or about 1200 mbar or more.

Thus, in some embodiments, at least a portion of the mixing procedure is carried out under oxygen-depleted conditions, such as under a positive pressure of inert gas (e.g. N₂ or Ar). Corticosteroid solutions manufactured according to the present invention may then be dispensed (filled) into suitable containers (bottles) for distribution to patients. The term “bottle” as used herein refers to any suitable container for dispensing corticosteroid solutions to patients. In particular, the term “bottle” encompasses vials and ampoules made from low density polyethylene (LDPE) or other pharmaceutically acceptable container material. In some embodiments, the filling procedure may be performed under oxygen-depleted conditions, e.g. under a blanket of an inert gas such as nitrogen or argon.

The filled pharmaceutically acceptable containers (e.g. vials or ampoules) may be packaged in pouches for distribution to patients. In general the number of pharmaceutically acceptable containers in each pouch will be a convenient number for dispensing to patients. Pouches will generally contain 1 to 20 pharmaceutically acceptable containers. In some preferred embodiments, the pouches contain 1 to 10 pharmaceutically acceptable containers. In some preferred embodiments, the number of pharmaceutically acceptable containers in each pouch is 1, 2, 3, 4, 5, 6, 7, 8, 9, 10, 11, 12, 13, 14 or more pharmaceutically acceptable containers. The pouches are advantageously sealed. In some embodiments, the pouches are made of oxygen-impermeable material in order to exclude atmospheric oxygen from the pouches. In some embodiments, the pouches may be sealed under oxygen-depleted conditions (e.g. under a positive pressure of nitrogen or argon).

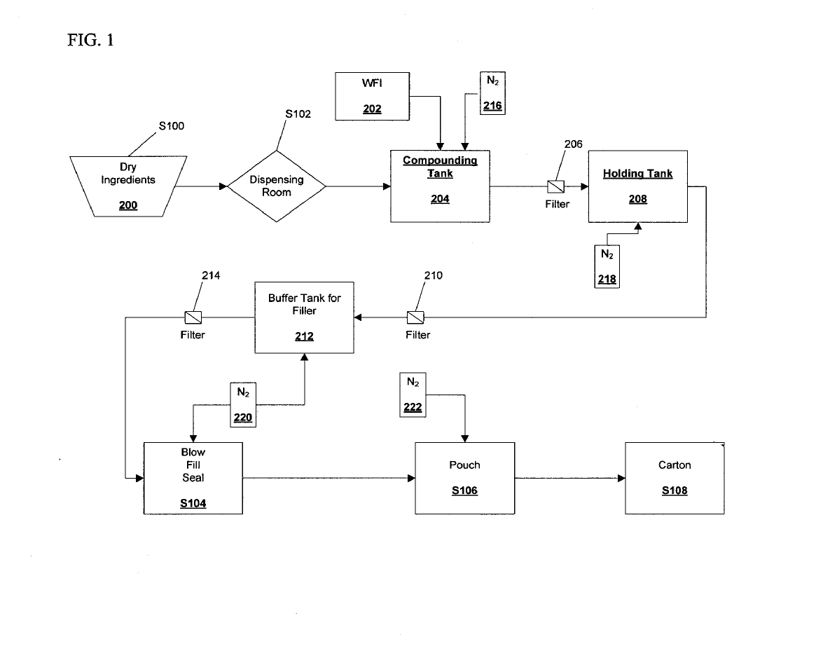

An illustrative, non-limiting example of a process according to the present invention is illustrated in FIG. 1. While certain process steps are illustrated in FIG. 1, in some embodiments not all the process steps are required. In S100, dry ingredients 200 are identified and are assayed to determine their water content. Dry ingredients 200 include corticosteroid (e.g. budesonide, and particularly micronized budesonide) and cyclodextrin (e.g. Captisol® cyclodextrin), as well as additional ingredients, such as citric acid, sodium citrate, sodium chloride and sodium EDTA (sodium edetate). In S102, the ingredients 200 are moved to a dispensing room and are weighed and placed in containers suitable for dispensing the ingredients into the compounding tank 204. The cyclodextrin is advantageously divided into three aliquots; and the corticosteroid (e.g. budesonide) is placed in a suitable container. Water for injection (WFI) 202 is charged into the compounding tank 204. The dry ingredients 200 are then added to the compounding tank 204. At least a portion of the mixing in the compounding tank 204 is conducted under oxygen-depleted conditions. For example, the WFI 202 may have been sparged with nitrogen or argon to remove dissolved oxygen. Alternatively, the compounding tank 204 may be sealed and subjected to one or more (preferably two) cycles of vacuum/hold/overpressure with inert gas 216 (such as nitrogen or argon) during the mixing process. The overpressure of inert gas 216 may be a value above atmospheric pressure (any positive gauge pressure), and may for example be in the range of from 100 mbar to about 3000 mbar. In currently preferred embodiments, the overpressure is about 1,200 mbar of nitrogen gas. In some embodiments, the compounding tank 204 is fitted with a homogenization apparatus that is designed to create high shear conditions. In some embodiments, the compounding tank 204 is a FrymaKoruma Dinex® compounding mixer, which comprises a holding tank with a water jacket, an inlet for introducing liquid ingredients (e.g. WFI), a homogenizer, a stirrer, a short loop, a long loop and a funnel for introducing dry ingredients. High shear conditions in the FrymaKoruma Dinex® compounding mixer are approximately 1000 rpm to 4000 rpm, preferably about 1500 rpm to about 3000 rpm. For the 500 L batch size in a compounding tank 204 designed to accommodate a maximum volume of 500 L, one preferred homogenizer speed is about 2,500 rpm, although other values may be selected by one having skill in the art. For a 50 L batch size in a compounding tank 204 designed to accommodate a maximum volume of 500 L, one preferred homogenizer speed is about 1,700 rpm, although other values may be selected by one having skill in the art. The compounding tank 204 may be sealed to exclude atmospheric gasses. The compounding tank 204 may be any suitable size, in particular about 50 L to 1000 L capacity. The 500 L model is currently preferred. At the end of mixing (e.g. 30 to 600 min, and preferably about 120 min.) the corticosteroid (e.g. budesonide) solution is discharged under pressure into a holding tank 208. In some embodiments, a filter 206 is located between the compounding tank 204 and the holding tank 208. The filter may be a 0.1 to 0.22 μm mean pore diameter filter (preferably a 0.22 μm mean pore diameter) of a suitable composition (e.g. PVDF), e.g. a Millipore® CVGL71 TP3 0.22 μm filter.

The corticosteroid (e.g. budesonide) solution may be held in the holding tank 208 for a period of time, e.g. up to seven days. The holding tank 208 may be air-tight and may be charged with an overpressure of inert gas 218, such as nitrogen or argon. In general, the inert gas pressure should be held well above atmospheric pressure, e.g. about 2000 mbar. The corticosteroid (e.g. budesonide) solution is next discharged under pressure into a buffer tank 212. The buffer tank 212 provides a mechanical buffer between the holding tank 208 and the filler in the Blow Fill Seal step S104. The buffer tank may also have a inert gas 220 overlay. A filter 210 may be interposed between the holding tank 208 and the buffer tank 212. When present, the filter 210 may be a 0.1 to 0.22 sum mean pore diameter filter (preferably a 0.22 μm mean pore diameter) of a suitable composition (e.g. PVDF), e.g. a Millipore® CVGL71TP3 0.22 μm filter.

The budesonide solution is discharged from the buffer tank 212 to a Blow Fill Seal apparatus in step S104. A filter 214 may be interposed between the buffer tank 212 and the Blow Fill Seal apparatus in step S104. When present, the filter 214 may be a 0.1 to 0.22 μm filter (preferably a 0.22 μm PVDF filter), e.g. a Millipore® CVGL71TP 3 0.22 μm filter. The Blow Fill Seal step S104 entails dispensing the liquid corticosteroid (e.g. budesonide) solution into individual pharmaceutically acceptable containers (referred to elsewhere herein as bottles, ampoules or vials) and sealing the individual containers. In some embodiments, the containers are LDPE ampoules having a nominal capacity of 0.5 ml, although other materials and sizes are within the skill in the art. In some embodiments, the Blow Fill Seal step S104 may be conducted under oxygen-depleted conditions, such as positive inert gas 220 (e.g. nitrogen) pressure. The individual containers are then packaged in pouches in the Pouch step S106. In some embodiments, the Pouch step S106 may be carried out under oxygen-depleted conditions, such as under positive inert gas 222 (e.g. nitrogen) pressure. Each pouch may contain one or more containers (e.g. ampoules or vials) of corticosteroid (e.g. budesonide). In some embodiments, each pouch contains 1, 2, 3, 4, 5, 6, 7, 8, 9, 10, 11, 12, 13, 14 or more containers. In some embodiments, a preferred number is 5 containers per pouch. The pouches are packaged into cartons in the Carton step S108.

Corticosteroid (e.g. budesonide) solutions prepared by methods according to the invention are used to treat one or more respiratory disorders. The corticosteroid solutions are advantageously compounded such that the active pharmaceutical ingredients contained therein are available on a unit dosage basis in a therapeutically effective amount. A therapeutically effective amount or effective amount is that amount of a pharmaceutical agent to achieve a pharmacological effect. The term “therapeutically effective amount” includes, for example, a prophylactically effective amount. An “effective amount” of a corticosteroid, such as budesonide, is an amount effective to achieve a desired pharmacologic effect or therapeutic improvement without undue adverse side effects. The effective amount of a corticosteroid, such as budesonide, will be selected by those skilled in the art depending on the particular patient and the disease level. It is understood that “an effective amount” or “a therapeutically effective amount” can vary from subject to subject, due to variation in metabolism of a corticosteroid, such as budesonide, age, weight, general condition of the subject, the condition being treated, the severity of the condition being treated, and the judgment of the prescribing physician.

The terms “treat” and “treatment” as used in the context of a bronchoconstrictive disorder refer to any treatment of a disorder or disease related to the contraction of the bronchia, such as preventing the disorder or disease from occurring in a subject which may be predisposed to the disorder or disease, but has not yet been diagnosed as having the disorder or disease; inhibiting the disorder or disease, e.g., arresting the development of the disorder or disease, relieving the disorder or disease, causing regression of the disorder or disease, relieving a condition caused by the disease or disorder, or stopping the symptoms of the disease or disorder. Thus, as used herein, the term “treat” is used synonymously with the term “prevent.”

Specific disorders that may be treated with compositions of the invention include, but are not limited to, respiratory diseases characterized by bronchial spasm, bronchial inflammation, increased phlegm viscosity, decreased lung capacity, etc. Specific conditions that may be treated include asthma, reactive airway disease and chronic obstructive pulmonary disease (COPD).

As used herein, the term “% impurity” and its related grammatical forms, means the fraction of impurities present in the corticosteroid solution in relation to the total active ingredients in the solution. In some embodiments, the % impurity may be measured by HPLC, with the % impurities being the total area of impurity peaks divided by the total of area of the active ingredient peaks and expressed as a percentage.

### EXAMPLES

### Example 1

### Preparation of 120 Microgram/Milliliter Budesonide Solution

A 50 L batch of budesonide solution (nominally 120 μg/ml) was prepared according to the following procedure:

Prior to weighing the Captisol® cyclodextrin (Cyclodextrin) and budesonide, the starting materials were assayed. The assay values were used to calculate the actual amount of Cyclodextrin and budesonide starting materials to be used in the formulation. The Cyclodextrin was found to be 4.9% water (95.1% Cyclodextrin). Thus, the total amount of Cyclodextrin starting material was increased by a proportional amount. It was calculated that the amount of Cyclodextrin starting material needed was 935.8569 g (representing 890.0 g Cyclodextrin). This Cyclodextrin starting material was weighed out in three measures: 735.86 g, 100.0 g and 100.0 g. In the same way, the budesonide starting material was assayed and found to contain 98.2% budesonide base. The amount of budesonide starting material was then calculated to be 5.95 g/0.982=6.06 g. Thus, 6.06 g of budesonide starting material was weighed out.

The following additional ingredients were weighed out: 15.0 g citric acid anhydrous; 25.0 g sodium citrate dihydrate USP. Sufficient water for injection to make up 50 kg of solution was also provided.

The mixing apparatus comprised a high sheer mixer a feed funnel in an isolator, as well as a vacuum apparatus and a source of nitrogen gas. The high sheer mixer was enclosed, thereby making it possible to apply a vacuum to the contents of the mixer during mixing.

Precisely 40 kg of water were introduced into to a mixing apparatus (FrymaKoruma Dinex® 700 vacuum processor (FrymaKoruma GmbH, Neuenburg, Del.), 500 L max volume). A 224 mbar vacuum was taken on the mixing apparatus and held for 5 minutes. Then 1278 mbar (gauge pressure) of nitrogen gas was introduced into the mixing vessel, which remained isolated from atmosphere outside the mixer during the duration of the mixing procedure. About one third of the Captisol® cyclodextrin was added to the funnel in the isolator. Then about 100.0 g of Cyclodextrin was added to the budesonide starting material in an Erlenmeyer flask and shaken until a homogeneous mixture was formed. This mixture was then added to the feed funnel. Then 100.0 g of Cyclodextrin was added to the Erlenmeyer flask and shaken until homogeneous. The contents of the Erlenmeyer flask were then added to the funnel. Finally 15.0 g citric acid anhydrous, 25.0 sodium citrate dihydrate USP, 5.0 g sodium EDTA dihydrate and 325.0 g sodium chloride were each sequentially added to the funnel. When all the ingredients had been combined in the funnel, all were introduced to the mixer by vacuum suction.

The contents of the mixer were then homogenized at 1500 rpm for about 5 minutes at about 17° C. The Erlenmeyer flask that formerly contained the budesonide starting material was then rinsed twice with about 150 ml water; and the rinse water was added to the funnel. Abut half of the remaining water was added to the funnel and the contents of the funnel were introduced into the mixer by vacuum suction. Then the final quantity of water was added to the funnel and introduced into the mixer by vacuum suction. Finally, the homogenizer speed was increased to 1700 rpm for 120 minutes.

During the 120 minute homogenization, the mixing tank was purged of oxygen as follows: (1) A first vacuum of about 200 mbar was applied and held for about 5 minutes; (2) a nitrogen pressure of 1200 mbar was applied; (3) a second vacuum of about 200 mbar was applied and held for about 5 minutes; and (4) a second nitrogen overlay of about 1215 mbar was applied to the mixer. At the end of homogenization, samples of the homogenized budesonide solution were taken and sent to Q.C.

### Example 2

### Sterilization Procedure

The homogenized budesonide solution from Example 1 was filtered through a 0.22 μm Millipore (CVGL71TP3) filter through a Teflon® PTFE hose into a sterilized holding tank. An overpressure of about 1200 mbar of nitrogen was applied to the filtered solution.

After the sterilized budesonide solution was collected in the holding tank, it was assayed. The budesonide solution was found to contain 98.2±0.5% of the theoretical concentration of budesonide, based upon the amount of budesonide in the budesonide starting material.

The sterilized budesonide solution is dispensed into pharmaceutically acceptable containers and sample pharmaceutically acceptable containers are tested for stability. The solution passed sterility according to USP <71> and PhEur 2.6.1.

### Example 3

### Stability of the Corticosteroid Composition

Sterilized budesonide solutions prepared according to procedures similar to those set forth in Examples 1 and 2 were dispensed into low density polyethylene (LDPE) ampoules under nitrogen and packaged in pouches of six ampoules each then put up on stability under accelerated conditions (i.e. subjected to 40° and 75% relative humidity). The results in Table 1 demonstrate that manufacturing the budesonide solution under oxygen-depleted conditions result in enhanced stability of the budesonide active ingredient. The starting budesonide (BUD) concentration is shown in the second colunn, the results of budesonide assays performed after 6 weeks at the indicated accelerated conditions appear in the third column and the results of budesonide assays performed after 3 months at the indicated accelerated conditions appear in the fourth column. Each solution passed sterility according to USP <71> and PhEur 2.6.1.

TABLE 1

| | BUD Start | BUD 6 weeks | BUD 3 months |
|---|---|---|---|
| Trial # | μg/ml/% | μg/ml/% | μg/ml/% |
| 1 | 230.62/100% | 229.66/99.58% | 230.86/100.1% |
| 2 | 236.92/100% | 235.77/99.5% | 235.93/99.6% |
| 3 | 235.1/100% | 233.87/99.5% | 236.67/100.7% |
| 4 | 236.77/100% | 236.01/99.7% | 234.66/99.1% |
| 5 | 121.13/100% | 120.27/99.3% | 119.96/99.03% |
| 6 | 119.62/100% | 117.97/98.6% | 119.43/99.8% |
| 7 | 240.23/100% | 236.23/98.3% | 237.16/98.7% |
| 8 | 241.2/100% | 241.79/100.3% | 239.52/99.3% |
| 9 | 119.32/100% | 118.91/99.7% | 119.45/100.1% |

### Example 4

### Additional Stability Studies for 240 and 120 Microgram/Milliliter Budesonide

Three batches each of 240 μg/ml and 120 μg/ml (nominal concentration) budesonide solutions were prepared essentially as described above, with mixing being performed under oxygen-depleted conditions (positive pressure nitrogen gas). The budesonide solutions were blow fill sealed in LDPE ampoules under nitrogen (0.5 ml nominal fill volume) and the ampoules were pouched under nitrogen (five ampoules per pouch). The pouched ampoules were then put up on stability. Each solution passed sterility according to USP <71> and PhEur 2.6.1.

Stability studies were conducted under the following conditions: Low Temperature (5° C.); Normal Conditions (25° C., 60% relative humidity); Accelerated Conditions (40° C., 75% relative humidity) and Intermediate Conditions (35° C., 65% relative humidity). The initial samples were assayed. Samples were also pulled at 3, 6, 9 and 12 months and assayed. The results of these stability studies are set forth in the following Table 2:

TABLE 2

| | | FI141 | FJ032A | FJ114 | FJ037 | FJ102 | FJ110 |
|---|---|---|---|---|---|---|---|
| Storage time | Batch/ | [μg/ml] | [μg/ml] | [μg/ml] | [μg/ml] | [μg/ml] | [μg/ml] |
| [months] | Conditions | (% of Start) | (% of Start) | (% of Start) | (% of Start) | (% of Start) | (% of Start) |
| Initial | 25° C./60% RH | 230.62 | 236.92 | 241.20 | 121.13 | 119.62 | 119.32 |
| | | (100%) | (100%) | (100%) | (100%) | (100%) | (100%) |
| 1.5 | 5° C. | 228.78 | 236.13 | 242.91 | 120.56 | 118.24 | 119.54 |
| | | (99.2%) | (99.7%) | (101%) | (99.5%) | (98.8%) | (100%) |
| | 25° C./60% RH | 229.60 | 236.33 | 242.94 | 120.93 | 118.44 | 119.15 |
| | | (99.6%) | (99.8%) | (101%) | (99.8%) | (99.0%) | (99.9%) |
| | 30° C./65% RH | 229.58 | 236.35 | 242.51 | 120.24 | 118.73 | 124.93 |
| | | (99.5%) | (99.8%) | (101%) | (99.3%) | (99.0%) | (105%) |
| | 40° C./75% RH | 229.66 | 235.77 | 241.79 | 120.27 | 117.97 | 118.91 |
| | | (99.5%) | (99.5%) | (100%) | (99.3%) | (98.6%) | (99.7%) |
| 3 | 5° C. | 230.73 | 236.65 | 243.18 | 121.01 | 119.84 | 119.94 |
| | | (100%) | (99.9%) | (101%) | (99.9%) | (100%) | (100%) |
| | 25° C./60% RH | 231.02 | 236.46 | 243.17 | 121.07 | 119.82 | 119.85 |
| | | (100%) | (99.8%) | (101%) | (100%) | (100%) | (100%) |
| | 30° C./65% RH | 230.61 | 236.69 | 242.86 | 121.35 | 119.63 | 120.00 |
| | | (100%) | (99.9%) | (101%) | (100%) | (100%) | (101%) |
| | 40° C./75% RH | 230.86 | 235.93 | 239.52 | 119.96 | 119.43 | 119.45 |
| | | (100%) | (99.6%) | (99.3%) | (99.0%) | (99.8%) | (100%) |
| 6 | 5° C. | 232.77 | 235.93 | 241.40 | 121.51 | 119.46 | N.T. |
| | | (101%) | (99.6%) | (100%) | (100%) | (99.9%) | (N/A) |
| | 25° C./60% RH | 233.26 | 235.89 | 242.00 | 121.00 | 119.51 | 118.32 |
| | | (101%) | (99.6%) | (100%) | (99.9%) | (99.9%) | (99.2%) |
| | 30° C./65% RH | 233.24 | N.T. | 239.06 | 120.86 | N.T. | N.T. |
| | | (101%) | (N/A) | (99.1%) | (99.8%) | (N/A) | (N/A) |
| | 40° C./75% RH | 231.96 | 234.29 | 240.32 | 118.56 | 117.98 | 118.97 |
| | | (101%) | (99.9%) | (99.6%) | (97.9%) | (98.6%) | (99.7%) |
| 9 | 5° C. | N.T. | 236.48 | 241.52 | 121.14 | 119.77 | N.T. |
| | | (N/A) | (99.8%) | (100%) | (100%) | (100%) | (N/A) |
| | 25° C./60% RH | 231.2 | 234.69 | 241.88 | 120.65 | 119.43 | 119.37 |
| | | (100%) | (99.1%) | (100%) | (99.6%) | (99.8%) | (100%) |
| | 30° C./65% RH | N.T. | N.T. | 239.07 | 120.62 | N.T. | N.T. |
| | | (N/A) | (N/A) | (99.1%) | (99.6%) | (N/A) | (N/A) |
| | 40° C./75% RH | N.T. | 232.91 | 239.77 | 119.4 | 118.00 | N.T. |
| | | (N/A) | (98.3%) | (99.4%) | (98.6%) | (98.6%) | (N/A) |
| 12 | 5° C. | N.T. | 235.46 | 244.61 | 122.53 | 120.88 | N.T. |
| | | (N/A) | (99.4%) | (101%) | (101%) | (101%) | (N/A) |
| | 25° C./60% RH | 233.4 | 238.19 | 245.48 | 121.47 | 120.28 | 121.15 |
| | | (101%) | (101%) | (102%) | (100%) | (100%) | (102%) |
| | 30° C./65% RH | N.T. | N.T. | 241.92 | 121.35 | N.T. | N.T. |
| | | (N/A) | (N/A) | (100%) | (100%) | (N/A) | (N/A) |
| | 40° C./75% RH | N.T. | 233.91 | 241.34 | 120.27 | 118.32 | N.T. |
| | | (N/A) | (98.7%) | (100%) | (99.3) | (98.9%) | (N/A) |

N.T. = Not Tested;
N/A = Not Applicable

As can be seen in the foregoing table, the method according to the present invention provides long-term stability for budesonide at 3, 6, 9 and 12 months and under Low Temperature, Normal, Intermediate and Accelerated conditions. In particular, the invention provides less than about 2% loss of budesonide potency up to 3, 6, 9 and 12 months under 5° C. conditions. The invention further provides less than about 2% loss of budesonide potency up to 3, 6, 9 and 12 months under 25° C., 60% relative humidity conditions. The invention also provides less than about 2% loss of budesonide potency up to 3, 6, 9 and 12 months under 35° C., 65% relative humidity. Moreover, the invention provides less than about 2% loss of budesonide potency up to 3, 6, 9 and 12 months under 40° C., 75% relative humidity. Thus the process of the invention provides for enhanced stability of budesonide solutions. It is expected from the 12 month, 40° C., 75% relative humidity data that budesonide solutions according to the invention will have less than 10% degradation in budesonide potency after 24 months at normal patient use conditions (i.e. 25° C., 60% relative humidity).

### Example 5

### Impurity Data for 240 and 120 Microgram/Milliliter Budesonide Solutions

Samples from the budesonide batches described in Example 4, above, were analyzed for impurities using HPLC detection. Impurity levels were calculated as the total area under the HPLC curve for all impurities divided by total area under the curve for the HPLC run and expressed in percentages (%). The results of the impurity analysis are set forth in the following tables 3A and 3B:

TABLE 3A

| | | Storage at 25° C./60% RH | | | | |
|---|---|---|---|---|---|---|
| Batch | Initial | 1.5 | 3 | 6 | 9 | 12 |
| FI141 | 0.32 | 0.33 | 0.41 | 0.59 | 0.63 | 0.66 |
| FJ032A | 0.37 | 0.33 | 0.44 | 0.55 | 0.49 | 0.23 |
| FJ114 | 0.35 | 0.4 | 0.4 | 0.39 | 0.47 | 0.56 |
| FJ037 | 0.58 | 0.51 | 0.58 | 0.66 | 0.93 | 0.93 |
| FJ102 | 0.4 | 0.4 | 0.39 | 0.47 | 0.59 | 0.6 |
| FJ110 | 0.41 | 0.39 | 0.43 | 0.48 | 0.53 | 0.77 |

TABLE 3B

| | | Storage at 40° C./75% RH | | | | |
|---|---|---|---|---|---|---|
| Batch | Initial | 1.5 | 3 | 6 | 9 | 12 |
| FI141 | 0.32 | 0.54 | 0.79 | 1.45 | N.T. | N.T. |
| FJ032A | 0.37 | 0.53 | 0.76 | 1.21 | 1.53 | 0.66 |
| FJ114 | 0.35 | 0.48 | 0.65 | 1.11 | 1.32 | 1.7 |
| FJ037 | 0.58 | 0.71 | 0.91 | 1.33 | 2.02 | 2.21 |
| FJ102 | 0.4 | 0.51 | 0.81 | 1.23 | 1.75 | 2.11 |
| FJ110 | 0.41 | 0.57 | 0.8 | 1.16 | N.T. | N.T. |

N.T. = Not Tested

As can be seen in the foregoing tables, the process according to the present invention provides excellent stability for budesonide solutions, as evidenced by the impurity levels in the foregoing tables.

### Example 6

### 80 Microgram/Milliliter Budesonide Solution (Batch G1059)

A 50 L batch of budesonide solution having a final concentration of approximately 80 μg/ml was prepared according to the following procedure.

First budesonide and Captisol® cyclodextrin (Cyclodextrin) were assayed to determine the percent water in each sample. The target mass of cyclodextrin in the 50 L batch was 595 g; and the target mass of budesonide was 4.1 g. The assay for Cyclodextrin gave a value of 4.8% water or 95.2% Cyclodextrin; the budesonide assay gave a percent budesonide value of 99.2%. Thus, the amount of Cyclodextrin was calculated to be 595 g/0.952=625 g Cyclodextrin; the budesonide mass was calculated to be 4.1 g/0.992=4.133 g budesonide.

The cyclodextrin was weighed out in three aliquots of 100 g, 100 g and 425 g of cyclodextrin, respectively. Precisely 4.133 g of budesonide were weighed out in a container (budesonide container).

A cleaned holding tank was steam sterilized and 40 kg of water for injection (WFI) were charged into the holding tank. A clean stainless steel 500 L (max capacity) FrymaKoruma Dinex® (FrymaKoruma GmbH, Neuenburg, Germany) mixing vessel (mixing tank) with a stirrer and homogenizer was steam sterilized for 10 minutes and dried. The mixing tank is equipped with a short homogenization loop (short loop) and a funnel for introduction of dry ingredients (dry-addition funnel; funnel). The 40 kg of water were then transferred to the mixing tank from the holding tank under pressure. Approximately half of the pre-weighed 425 g aliquot of Cyclodextrin were then added to the dry-addition funnel. The entire contents of the budesonide container were then added to the funnel, taking care not to allow any of the budesonide to contact the walls of the funnel. The first 100 g aliquot of Cyclodextrin was then added to the budesonide container and shaken to scavenge any residual budesonide. The contents of the budesonide container were then added to the funnel. This procedure was repeated with the second 100 g aliquot of Cyclodextrin.

The following quantities of ingredients were then added to the funnel: 15.0 of anhydrous citric acid, 25.0 g of sodium citrate dihydrate, 5.0 g sodium edetate dihydrate, 395.0 g of sodium chloride and the second half of Cyclodextrin from the 425 g aliquot. With the stirrer set to 25 rpm and the homogenizer set to 1500 rpm, the entire contents of the dry funnel were added to the mixing tank under suction. The contents of the mixing tank were then homogenized through the short loop for approximately 10 minutes.

The budesonide container was then washed with two 150 g aliquots of WFI: A first 150 g aliquot of WFI was added to the budesonide container and shaken. The contents of the budesonide container were then added to the funnel. This procedure was repeated with a second 150 g aliquot of WFI and then the entire contents (˜300 ml) of the funnel were added to the mixing tank by suction. Approximately half of 8.631 kg of WFI was added to the funnel. The WFI in the funnel was then added to the mixing tank by suction. This procedure was repeated with the remaining approximately half of the 8.631 kg of WFI.

The homogenizer speed was increased to 1700 rpm. The mixing tank was then purged with nitrogen (N₂): A vacuum of −200 mbar was applied to the mixing tank and held for five minutes; then the mixing tank was pressurized with 1,200 mbar of nitrogen. This procedure was repeated once. Samples of budesonide solution were drawn from the mixing tank through a 0.22 μm PVDF filter at 60, 90 and 120 minutes. At the end of 124 minutes, the entire contents of the mixing tank were discharged through Teflon® PTFE hose and a 0.22 μm Durapore® PVDF cartridge filter and into a holding tank. The procedure netted 46.6 kg of 80.2 μg/ml (assay value) budesonide solution. The budesonide solution was blow filled into LDPE vials under nitrogen to produce filled vials containing 0.53 ml/vial (42.1 μg/vial of budesonide). The sealed LDPE vials were pouched—five vials per pouch—under nitrogen.

### Example 7

### Additional Budesonide Compositions

Budesonide compositions were prepared essentially as described above. The Table 4, below, provides the ingredients for four different concentrations of budesonide solution according to the present invention.

TABLE 4

| | 240 mcg/ | 120 mcg/ | 60 mcg/ | 40 mcg/ |
|---|---|---|---|---|
| Ingredient | 0.5 mL | 0.5 mL | 0.5 mL | 0.5 mL |
| Budesonide | 0.048 | 0.024 | 0.012 | 0.008 |
| Captisol | 7.5 | 3.57 | 1.78 | 1.19 |
| Citric acid | 0.03 | 0.03 | 0.03 | 0.03 |
| Sodium Citrate | 0.05 | 0.05 | 0.05 | 0.05 |
| Dihydrate USP | | | | |
| NaCl | 0.45 | 0.57 | 0.73 | 0.79 |
| Na-EDTA | 0.01 | 0.01 | 0.01 | 0.01 |
| Water | ad 100.0 | ad 100.0 | ad 100.0 | ad 100.0 |

### Example 8

### Open Pouch Stability

In order to evaluate the stability of budesonide solutions according to the present invention under patient use conditions, open pouch stability studies were performed. Budesonide ampoules are sealed in air-tight pouches under nitrogen pressure. Generally multiple ampoules are packaged in each pouch and the patient is instructed to open the pouch, use as many budesonide ampoules as are prescribed for the patient's condition, return the remaining ampoules to the pouch, and place it in a convenient location for future use. Under these conditions, the first ampoules to be taken from the pouch will have been under a nitrogen atmosphere since the pouch was sealed, while the later-used ampoules will have been exposed to a normal atmospheric mixture of oxygen and nitrogen for a period of time between when the pouch was opened and when the ampoules were used. In order to determine whether opening the pouch would have any significant impact on the purity of the budesonide solution over time, open pouch patient use conditions were simulated in an open pouch stability study.

Budesonide solution (240 μg/ml) in 0.5 ml LDPE ampoules was prepared essentially as set forth above. Mixing, Blow-Fill-Seal and Pouching operations were conducted under oxygen-depleted conditions. In particular, mixing was carried out with two cycles of vacuum (−200 mbar) followed by 1200 mbar of nitrogen overlay. Blow-Fill-Seal and pouching were carried out under a nitrogen blanket. After pouching the ampoules, sample pouches were randomly selected from the batch and were opened, thereby allowing ambient atmosphere to replace the nitrogen in the pouches. Ampoules were tested for impurities using an HPLC impurity assay and non-budesonide peaks were identified as “impurities.” Total impurities were calculated as the total area under the curve for all HPLC impurity peaks divided by the total area under the HPLC curve and converted to percentages. Ampoules were tested immediately after the pouches were opened (t=0). The remaining pouches were put up on stability at 25° C. and 60% relative humidity, with sample ampoules analyzed by HPLC impurity assay at 2 weeks, 4 weeks and 8 weeks after the pouches were opened. The results of these experiments are set forth in Table 5, below:

TABLE 5
Open-Pouch Stability Data [240 μg/ml] at 25° C.

| | 0 | 2 wk | 4 wk | 8 wk |
|---|---|---|---|---|
| HP004 Budesonide | 236.92 | 235.37 | 235.06 | N.T. |
| Assay [μg/mL] | | | | |
| Total Impurities [Area | 0.37 | 0.42 | 0.35 | N.T. |
| %] | | | | |
| HP022 Budesonide | 230.45 | N.T. | 230.26 | 229.41 |
| Assay [μg/mL] | | | | |
| Total Impurities [Area | 0.44 | N.T. | 0.5 | 0.86 |
| %] | | | | |

N.T. = not tested

As can be seen from the foregoing table, the budesonide solution of the present invention demonstrate remarkable stability in open pouch stability tests. Both tested batches of 240 μg/ml budesonide solution demonstrated 0.5% or less impurity concentrations at the 4 week time point, and one of the budesonide solutions demonstrated less than 0.9% impurities after 8 weeks of exposure to ambient air pressure. This demonstrates the ability of the invention to prepare budesonide solutions in a form having long-term stability in normal patient use conditions.

### Example 9

### 40, 60, 120 and 240 μg/0.5 mL Dose Budesonide Solutions

Following the general procedures outlined in Examples 1 and 6, above, budesonide solutions having concentrations of 80, 120, 240 and 480 μg/mL were prepared, dispensed into LDPE vials (ampoules) in 0.5 mL doses and pouched as described above. The resulting 0.5 mL doses contained 40, 60, 120 and 240 μg budesonide per 0.5 mL dose. The amounts of each ingredient contained in each ampoule are set forth in Table 6, below.

TABLE 6
40, 60, 120 and 240 μg/0.5 mL Dose Budesonide

| | 240 μg/ | 120 μg/ | 60 μg/ | 40 μg/ |
|---|---|---|---|---|
| Ingredient | 0.5 mL | 0.5 mL | 0.5 mL | 0.5 mL |
| Budesonide | 0.048 | 0.024 | 0.012 | 0.008 |
| Captisol | 7.5 | 3.57 | 1.78 | 1.19 |
| Citric acid | 0.03 | 0.03 | 0.03 | 0.03 |
| Sodium Citrate | 0.05 | 0.05 | 0.05 | 0.05 |
| Dihydrate USP | | | | |
| NaCl | 0.45 | 0.57 | 0.73 | 0.79 |
| Na-EDTA | 0.01 | 0.01 | 0.01 | 0.01 |
| Water | ad 100.0 | ad 100.0 | ad 100.0 | ad 100.0 |

Values shown are [w %]; Osmolality adjusted to 290 mOsm/kg; pH 4.5

### Example 10

### Aerosol Performance of Budesonide Solutions (60 μg/0.5 mL; 120 μ/0.5 mL)

Budesonide solutions having concentrations of 120 μg/mL (HP005: 60 μg/0.5 mL dose) and 240 μg/mL (HP011: 120 μg/0.5 mL dose) were prepared and filled essentially per methods described in Examples 1 and 6 above, with appropriate adjustments of concentrations of Cyclodextrin and budesonide (micronized). The aerosol stability of these solutions was characterized by breath simulation and Andersen Cascade Impactor (ACI) at time points of 0 months (Start: 0 M); 3 months (3 M); 6 months (6 M) and 9 months (9 M) after manufacturing of the budesonide solutions. The results of these studies are shown in Table 7, below. The delivered dose is the percentage of budesonide ejected from the nebulizer (PARI eFlow®, PARI GmbH, Munich, Del.) that is delivered to the lung. The Anderson Cascade Impactor (ACI), measures the Mass Median Aerodynamic Diameter (MAAD), the geometric standard deviation (GSD) and the percent of particles under 5 μm (respirable fraction).

TABLE 7
Aerosol Stability of Budesonide Solutions (60 μg/0.5 mL dose
and 120 μg/0.5 mL dose)

| | HP005 [60 mcg/0.5 mL] | | | | HP011 [120 mcg/0.5 mL] | | | |
|---|---|---|---|---|---|---|---|---|
| | 0 M | 3 M | 6 M | 9 M | 0 M | 3 M | 6 M | 9 M |
| Breath | | | | | | | | |
| Simulation | | | | | | | | |
| Delivered | 59.7 | 57.6 | 60.9 | 59.4 | 60.8 | 60.8 | 62.1 | 59.9 |
| Dose [%] | | | | | | | | |
| Recovery [%] | 97.1 | 95.1 | 96.2 | 96.3 | 98.7 | 97.0 | 96.6 | 96.7 |
| ACI | | | | | | | | |
| [28.3 L/min] | | | | | | | | |
| MMAD [um] | 3.4 | 3.3 | 3.3 | 3.2 | 3.5 | 3.2 | 3.3 | 3.1 |
| GSD | 1.4 | 1.4 | 1.4 | 1.4 | 1.4 | 1.4 | 1.4 | 1.4 |
| %<5 um | 88.0 | 88.3 | 88.6 | 90.0 | 87.8 | 89.9 | 88.8 | 90.8 |

MMD = Mass Median

As can be seen from the Table 7 above, budesonide solutions manufactured by methods according to the present invention possess remarkable aerosol stability for periods up to 9 months after manufacturing.

### Example 11

### Filling and Pouching Under Nitrogen and Air

The effect of filling and/or pouching sealed budesonide vials (ampoules) an air atmosphere, as opposed to a nitrogen atmosphere, several batches of budesonide were prepared, essentially as described in Example 6, above, except that in some batches air was substituted for nitrogen in the Blow-Fill-Seal step, the Pouching step or both. These batches were analyzed for stability. Stability was determined under 25° C./60% relative humidity and at 40° C./75% relative humidity at start (0 months), 3 months and 6 months after the budesonide was manufactured. Group 1 batches were compounded (mixed), dispensed (blow-filled and sealed) and pouched under nitrogen. Group 2 batches were compounded (mixed) and dispensed (blow-filled and sealed) under nitrogen and pouched under air. The Group 3 batch was compounded under nitrogen, dispensed under air and pouched under nitrogen. The group 4 batch was compounded under nitrogen, dispensed under air and pouched under air. Finally, the group 5 batch was compounded, dispensed and pouched under air. The results of these studies are set forth in Table 8 below.

TABLE 8
Stability of Budesonide Solutions Manufactured Under Oxygen-Depleted and Non-Oxygen Depleted
Conditions.

| | | | 3 m - | | 3 m - | | 6 m - | | 6 m - | | | | | |
|---|---|---|---|---|---|---|---|---|---|---|---|---|---|---|
| Lot | Start | | 25° C. | | 40° C. | | 25° C. | | 40° C. | | | | | |
| No. | Abs. | Δ | Abs | Δ | Abs | Δ | Abs | Δ | Abs | Δ | Gp | Cmp | Fill | Pch |
| FJ037 | 0.60 | 0.00 | 0.62 | 0.02 | 0.91 | 0.31 | 0.70 | 0.10 | 1.31 | 0.71 | 1 | N₂ | N₂ | N₂ |
| FJ102 | 0.42 | 0.00 | 0.39 | −0.03 | 0.80 | 0.38 | 0.49 | 0.07 | 1.22 | 0.80 | 1 | N₂ | N₂ | N₂ |
| FJ110 | 0.44 | 0.00 | 0.46 | 0.02 | 0.77 | 0.33 | 0.46 | 0.02 | 1.13 | 0.69 | 1 | N₂ | N₂ | N₂ |
| FI141 | 0.34 | 0.00 | 0.43 | 0.09 | 0.77 | 0.43 | 0.62 | 0.28 | 1.40 | 1.06 | 1 | N₂ | N₂ | N₂ |
| FJ032 | 0.38 | 0.00 | 0.47 | 0.09 | 0.76 | 0.38 | 0.58 | 0.20 | 1.23 | 0.85 | 1 | N₂ | N₂ | N₂ |
| FJ114 | 0.42 | 0.00 | 0.42 | 0.00 | 0.63 | 0.21 | 0.43 | 0.01 | 1.10 | 0.68 | 1 | N₂ | N₂ | N₂ |
| GB098 | 0.29 | 0.00 | 0.40 | 0.11 | 0.69 | 0.40 | 0.52 | 0.23 | 1.07 | 0.78 | 1 | N₂ | N₂ | N₂ |
| FJ097 | 0.37 | 0.00 | 0.43 | 0.06 | 0.68 | 0.31 | 0.55 | 0.18 | 1.07 | 0.70 | 1 | N₂ | N₂ | N₂ |
| FJ113 | 0.41 | 0.00 | 0.49 | 0.08 | 0.75 | 0.34 | 0.50 | 0.09 | 1.10 | 0.69 | 1 | N₂ | N₂ | N₂ |
| FJ032B | 0.39 | 0.00 | 0.46 | 0.07 | 0.81 | 0.42 | 0.51 | 0.12 | 1.19 | 0.80 | 2 | N₂ | N₂ | Air |
| GB098 | 0.31 | 0.00 | 0.43 | 0.12 | 0.77 | 0.46 | 0.50 | 0.19 | 1.31 | 1.00 | 2 | N₂ | N₂ | Air |
| “10 d” | | | | | | | | | | | | | | |
| GB111 | 0.33 | 0.00 | 0.38 | 0.05 | 0.71 | 0.38 | 0.54 | 0.21 | 1.22 | 0.89 | 3 | N₂ | Air | N₂ |
| GB111 | 0.30 | 0.00 | 0.46 | 0.16 | 0.87 | 0.57 | 0.64 | 0.34 | 1.44 | 1.14 | 4 | N₂ | Air | Air |
| “w/o N₂” | | | | | | | | | | | | | | |
| GB131 | 0.40 | 0.00 | 0.53 | 0.13 | 1.04 | 0.64 | 0.73 | 0.33 | 2.03 | 1.63 | 5 | Air | Air | Air |

Abs. = Area-percent of impurities in budesonide formulations at Start (0 m), 3 months (3 m), or 6 months (6 m)
Δ = Change in area-percent from Start time point
Gp = Group No.
Cmp = Compounding Step;
Fill = Blow Fill Seal Step;
Pch = Pouching Step

As can be seen above, mixing budesonide solution under oxygen-depleted atmosphere (e.g. nitrogen atmosphere) resulted in enhanced budesonide stability at 3 and 6 months under accelerated conditions (40° C./75% relative humidity) after manufacturing of the budesonide solution. Pouching of budesonide-filled vials (ampoules) under oxygen-depleted conditions also demonstrated statistically significant improvement in stability over pouching of budesonide-filled vials under air.

Although preferred embodiments of the present invention have been shown and described herein, it will be apparent to those skilled in the art that such embodiments are provided by way of example only. Numerous variations, changes, and substitutions will be apparent to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered herein.

What is claimed is:

1. A process of preparing a corticosteroid mixture, comprising mixing ingredients of the corticosteroid mixture in a mixing vessel under oxygen-depleted conditions to produce the corticosteroid mixture, wherein the ingredients include as starting materials corticosteroid and water.

2. The process of claim 1, further comprising storing the corticosteroid mixture in a holding tank.

3. The process of claim 2, wherein the corticosteroid is stored in the holding tank under oxygen depleted conditions.

4. The process of claim 3, further comprising dispensing the corticosteroid mixture into pharmaceutically acceptable containers.

5. The process of claim 4, wherein the corticosteroid mixture is dispensed into pharmaceutically acceptable containers under oxygen-depleted conditions.

6. The process of claim 1, further comprising dispensing the corticosteroid mixture into pharmaceutically acceptable containers.

7. The process of claim 6, wherein the corticosteroid mixture is dispensed into pharmaceutically acceptable containers under oxygen-depleted conditions.

8. The process of claim 6, further comprising packaging the pharmaceutically acceptable containers in one or more pouches.

9. The process of claim 8, wherein packaging the pharmaceutically acceptable containers in one or more pouches is carried out under oxygen-depleted conditions.

10. The process of claim 8, wherein the dispensing and pouching conditions are selected from one of the following: the corticosteroid is dispensed into pharmaceutically acceptable containers under oxygen-depleted conditions and the pharmaceutically acceptable containers are packaged in one or more pouches under oxygen-depleted conditions; the corticosteroid is dispensed into pharmaceutically acceptable containers under air and the pharmaceutically acceptable containers are packaged in one or more pouches under oxygen-depleted conditions; the corticosteroid is dispensed into pharmaceutically acceptable containers under oxygen-depleted conditions and the pharmaceutically acceptable containers are packaged in one or more pouches under air; and the corticosteroid is dispensed into pharmaceutically acceptable containers under air and the pharmaceutically acceptable containers are packaged in one or more pouches under air.

11. The process of claim 10, wherein the corticosteroid mixture contains less than about 0.5% impurities when one or more pouches are opened and the pharmaceutically acceptable containers are exposed to normal atmosphere for two weeks or less.

12. The process of claim 10, wherein the corticosteroid mixture contains less than about 1.0% impurities when one or more pouches are opened and the pharmaceutically acceptable containers are exposed to normal atmosphere for four weeks or less.

13. The process of claim 1, wherein the corticosteroid mixture is a solution.

14. The process of claim 1, wherein the corticosteroid mixture further comprises a solubility enhancer.

15. The process of claim 14, wherein the solubility enhancer is a sulfoalkyl ether cyclodextrin (SAE-CD).

16. The process of claim 15, wherein the solubility enhancer is SBE7-β-CD.

17. The process of claim 1, wherein the corticosteroid is budesonide.

18. The process of claim 1, wherein the cortitosteroid solution farther comprises an additional active pharmaceutical ingredient.

19. The process of claim 18, wherein the additional active pharmaceutical ingredient is a short acting β₂ agonist.

20. The process of claim 19, wherein the short acting β₂ agonist is albuterol.

21. The process of claim 1, wherein the oxygen-depleted conditions include one or more of: sparging the water, the mixture or both with inert gas; applying inert gas over the water, the mixture or both; or applying a vacuum to the water, the mixture or both.

22. A stable corticosteroid composition prepared by the process of claim 21.

23. The process of claim 21, wherein the inert gas is selected from nitrogen gas (N₂), argon gas (Ar) and mixtures thereof.

24. A stable corticosteroid composition prepared by the process of claim 23.

25. A corticosteroid mixture which loses no more than about 2% of corticosteroid potency after exposing the corticosteroid mixture to accelerated conditions of 40° C. and 75% relative humidity for a stability testing period of at least about 3 months.

26. The corticosteroid mixture of claim 25, wherein the stability testing period is at least about 6 months.

27. The corticosteroid mixture of claim 25, wherein the stability testing period is at least about 9 months.

28. The corticosteroid mixture of claim 25, wherein the stability testing period is about 12 months.

29. The mixture of claim 25 in the form of a solution.

30. The mixture of claim 25, wherein the corticosteroid mixture loses no more than 10% corticosteroid potency after 24 months at 25° C. and 60% relative humidity.

31. The mixture of claim 25, wherein the corticosteroid mixture further comprises a solubility enhancer.

32. The mixture of claim 31, wherein the solubility enhancer is a sulfoalkyl ether cyclodextrin (SAE-CD).

33. The mixture of claim 32, wherein the solubility enhancer is SBE7-β-CD.

34. The mixture of claim 25, wherein the corticosteroid is budesonide.

35. The mixture of claim 25, wherein the corticosteroid mixture further comprises an additional active pharmaceutical ingredient.

36. The mixture of claim 35, wherein the additional active pharmaceutical ingredient is a short acting β₂ agonist.

37. The mixture of claim 36, wherein the short acting β₂ agonist is albuterol.

38. The mixture of claim 25, wherein the mixture is produced by a process comprising mixing ingredients of the corticosteroid mixture in a mixing vessel under oxygen-depleted conditions to produce the corticosteroid mixture, wherein the ingredients include as starting materials corticosteroid and water.

39. The mixture of claim 38, wherein the process further comprises storing the corticosteroid mixture in a holding tank.

40. The mixture of claim 39, wherein the process further comprises storing the mixture in the holding tank under oxygen-depleted conditions

41. The mixture of claim 38, wherein the process further comprises dispensing the corticosteroid mixture into pharmaceutically acceptable containers.

42. The mixture of claim 41, wherein the corticosteroid mixture is dispensed into pharmaceutically acceptable containers under oxygen-depleted conditions.

43. The mixture of claim 42, wherein the pharmaceutically acceptable container are further packaged in pharmaceutically acceptable pouches.

44. The mixture of claim 43, wherein packaging the pharmaceutically acceptable containers in pouches is carried out under oxygen-depleted conditions.

45. A corticosteroid mixture which contains no more than 1.0% of impurities after exposing the corticosteroid mixture to accelerated conditions of 40° C. and 75% relative humidity for a stability testing period of at least about 3 months.

46. The corticosteroid mixture of claim 45, wherein the stability testing period is at least about 6 months.

47. The corticosteroid mixture of claim 45, wherein the mixture contains less than about 1.5% impurities after a stability testing period of at least about 9 months.

48. The corticosteroid mixture of claim 45, wherein the mixture contains less than about 2.5% impurities after a stability testing period of about 12 months.

49. The mixture of claim 45 in the form of a solution.

50. The mixture of claim 45, wherein the corticosteroid mixture further comprises a solubility enhancer.

51. The mixture of claim 50, wherein the solubility enhancer is a sulfoalkyl ether cyclodextrin (SAE-CD).

52. The mixture of claim 51, wherein the solubility enhancer is SBE7-β-CD.

53. The mixture of claim 45, wherein the corticosteroid is budesonide.

54. The mixture of claim 45, wherein the corticosteroid mixture further comprises an additional active pharmaceutical ingredient.

55. The mixture of claim 54, wherein the additional active pharmaceutical ingredient is a short acting β₂ agonist.

56. The mixture of claim 55, wherein the short acting β₂ agonist is albuterol.

57. The mixture of claim 45, wherein the mixture is produced by a process comprising mixing ingredients of the corticosteroid mixture in a mixing vessel under oxygen-depleted conditions to produce the corticosteroid mixture, wherein the ingredients include as starting materials corticosteroid and water.

58. The mixture of claim 57, wherein the process further comprises storing the corticosteroid mixture in a holding tank.

59. The mixture of claim 58, wherein the process further comprises storing the mixture in the holding tank under oxygen-depleted conditions

60. The mixture of claim 58, wherein the process further comprises dispensing the corticosteroid mixture into pharmaceutically acceptable containers.

61. The mixture of claim 60, wherein the corticosteroid mixture is dispensed into pharmaceutically acceptable containers under oxygen-depleted conditions.

62. The mixture of claim 61, wherein the pharmaceutically acceptable container are further packaged in pharmaceutically acceptable pouches.

63. The mixture of claim 62, wherein packaging the pharmaceutically acceptable containers in pouches is carried out under oxygen-depleted conditions.

64. The mixture of claim 25, wherein the budesonide solution demonstrates stable aerosol performance for at least 3 months after it is manufactured.

65. The process of claim 64, wherein the budesonide solution demonstrates stable aerosol performance for at least 6 months after it is manufactured.

66. The process of claim 65, wherein the budesonide solution demonstrates stable aerosol performance for at least 9 months after it is manufacture.

67. The process of claim 1, wherein the budesonide solution demonstrates stable aerosol performance for at least 3 months after it is manufactured.

68. The process of claim 67, wherein the budesonide solution demonstrates stable aerosol performance for at least 6 months after it is manufactured.

69. The process of claim 64, wherein the budesonide solution demonstrates stable aerosol performance for at least 9 months after it is manufacture.

70. The mixture of claim 25, wherein the corticosteroid solution loses no more than about 10% of corticosteroid potency after 24 hours of exposure to conditions of 25° C. and 60% relative humidity.
